(19) United States
(12) Patent Application Publication
(10) Pub. No.: US 2022/0194982 A1
(43) Pub. Date: Jun. 23, 2022

(54) PEPTIDES AND PEPTIDE CONJUGATES FOR TREATING MENTAL DISORDERS

(71) Applicants: Kyoto University, Kyoto-shi (JP); Kazusa DNA Research Institute, Kisrazu-shi (JP)

(72) Inventors: Kousaku Ohinata, Kyoto-shi (JP); Yukiha Mori, Kyoto-shi (JP); Hideyuki Suzuki, Kisarazu-shi (JP)

(73) Assignees: Kyoto University, Kyoto-shi, Kyoto (JP); Kazusa DNA Research Institute, Kisarazu-shi, Chiba (JP)

(21) Appl. No.: 17/686,748

(22) Filed: Mar. 4, 2022

Related U.S. Application Data
(62) Division of application No. 16/329,062, filed on Feb. 27, 2019.
(60) Provisional application No. 62/384,333, filed on Sep. 7, 2016.

(51) Int. Cl. C07K 7/06; A23L 33/18; A61K 47/54; A61K 47/56; A61P 25/22; A61P 25/00; A61K 9/00; A61K 9/50; C07K 7/08

(52) U.S. Cl. CPC C07K 7/06; A23L 33/18; A61K 47/543; A61K 47/548; A61K 47/56; A61K 47/54; A61K 38/00; A61P 25/22; A61P 25/00; A61K 9/0056; A61K 9/50; C07K 7/08; A23V 2002/00; A61K 47/549

(57) ABSTRACT

Peptides, salts thereof, peptide conjugates, and salts thereof having 5 to 15 amino acids and having an amino acid sequence comprising at least 5 consecutive amino acids from the amino acid sequence LSSTQAQQSX₁ (SEQ ID NO:34).

### 1. CROSS-REFERENCE TO RELATED APPLICATIONS

This application is a divisional of U.S. application Ser. No. 16/329,062, which is a national stage entry of international application no. PCT/JP2017/032079, filed Sep. 6, 2017, which claims the priority benefit of U.S. provisional application No. 62/384,333, filed Sep. 7, 2016, the contents of each of which are incorporated herein in their entireties by reference thereto.

### SEQUENCE LISTING

The instant application contains a Sequence Listing which has been submitted electronically in ASCII format and is hereby incorporated by reference in its entirety. Said ASCII copy, created on 25 Feb. 2019, is named P17-149WO_ST25.txt and is 9 Kilobytes in size.

### Background Art

### 2. BACKGROUND

It is estimated that 9.5% of the adult population of the United States suffers from a mood disorder (Kessler et al., 2005, Arch Gen Psychiatry 62(6):617-627). Only about one-half of those suffering from a mood disorder receive treatment, and less than 40% of those receiving treatment receive minimally adequate treatment (Wang et al., 2005, Arch Gen Psychiatry 62(6):629-640).

It is also estimated that 18.1% of the adult population of the United States suffers from an anxiety disorder (Kessler et al., 2005, Arch Gen Psychiatry 62(6):617-627). Less than 40% of those suffering from an anxiety disorder receive treatment, and less than 35% of those receiving treatment receive minimally adequate treatment (Wang et al., 2005, Arch Gen Psychiatry 62(6):629-640).

Disorders of diminished motivation occur frequently in individuals with traumatic brain injury, with estimates for the frequency varying from 5% to 67% (Marin and Wilkosz, 2005, J Head Trauma Rehabil 20(4):377-388). Diminished motivation is also often found in individuals suffering from depression, Parkinson's disease, Alzheimer's disease, and schizophrenia, as well as individuals who have suffered a stroke, and even in healthy individuals, particularly the elderly (Bonnelle et al., 2015, Journal of Physiology 109:16-26).

### SUMMARY OF INVENTION

### Technical Problem

New treatments for mood disorders, anxiety disorders, and disorders of diminished motivation are needed.

### Solution to Problem

### 3. SUMMARY

PCT international application no. PCT/JP2016/056453, the contents of which are incorporated herein by reference in their entireties, describes 10 amino acid peptides derived from the soybean storage protein β-conglycinin having amino acid sequences LSSTQAQQSY (SEQ ID NO:1), LSSTQAQQSW (SEQ ID NO: 6) and LSSTQAQQSF (SEQ ID NO:7), and their usefulness for treating conditions such as depression and decreased motivation. This disclosure provides peptides, salts thereof, peptide conjugates, and salts thereof (sometimes collectively referred to herein as “compounds”), which are 5 to 15 amino acids in length and comprise at least 5 consecutive amino acids in common with one of the 10 amino acid peptides described in PCT international application no. PCT/JP2016/056453.

In one aspect, the disclosure provides peptides and salts thereof that are 5 to 15 amino acids in length and have an amino acid sequence comprising at least 5 consecutive amino acids from the amino acid sequence LSSTQAQQSX₁ (SEQ ID NO:34), where X₁ is Y, W, of F. In certain embodiments, the peptides are 5-8, 6-10, 7-11, 8-12, 9-13, 10-14, 11-15 or 12-15 amino acids in length. Exemplary peptides and salts thereof are described in Section 5.1 and numbered embodiments 1 to 45 and 113 to 117 below.

In another aspect, the disclosure provides peptide conjugates and salts thereof comprising a peptide moiety attached to one or more (e.g., one, two or three) conjugate moieties. The peptide moiety can be 5 to 15 amino acids in length and comprise at least 5 consecutive amino acids from the amino acid sequence LSSTQAQQSX₁ (SEQ ID NO:34), where X₁ is Y, W, of F. In certain embodiments, the peptide moieties are 5-8, 6-10, 7-11, 8-12, 9-13, 10-14, 11-15 or 12-15 amino acids in length. Exemplary peptide conjugates and salts thereof are described in Section 5.2 and numbered embodiments 46 to 117 below.

The disclosure also provides pharmaceutical compositions and food products comprising a compound of the disclosure. Exemplary pharmaceutical compositions are described in Section 5.3 and numbered embodiment 118 below, and exemplary food products (e.g., dietary supplements and functional foods) are described in Section 5.4 and numbered embodiments 119 to 121 below.

The disclosure also provides methods for treating or preventing a mood disorder, an anxiety disorder, or a disorder of diminished motivation using the compounds, pharmaceutical compositions, and food products described herein. Exemplary methods of treating or preventing mood disorders, anxiety disorders, and disorders of diminished motivation are described in Section 5.5 and numbered embodiments 122 to 180 below.

### Brief Description of Drawings

### 4. BRIEF DESCRIPTION OF THE FIGURES

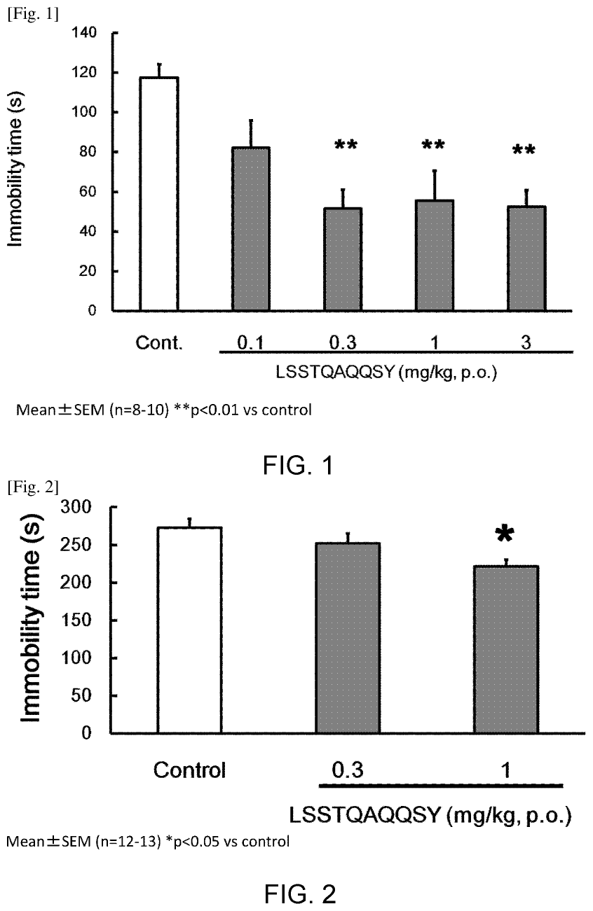

FIG. 1 shows the results of a tail suspension test in mice orally administered the peptide LSSTQAQQSY (SEQ ID NO:1).

FIG. 2 shows the results of a forced swimming test in mice orally administered the peptide LSSTQAQQSY (SEQ ID NO:1).

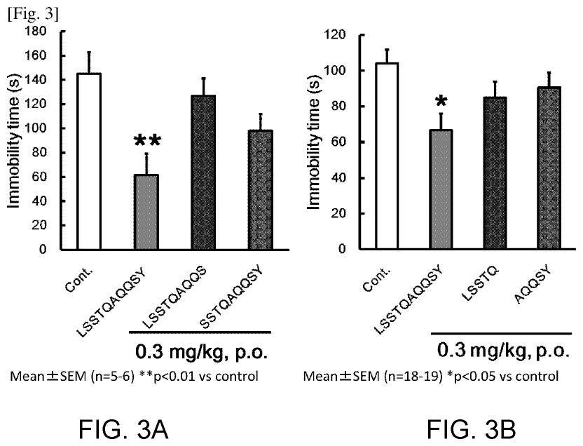

FIGS. 3A-3B show the results of a tail suspension test in mice orally administered the peptide LSSTQAQQSY (SEQ ID NO:1), LSSTQAQQS (SEQ ID NO:2), or SSTQAQQSY (SEQ ID NO:3) (FIG. 3A), or LSSTQAQQSY (SEQ ID NO:1), LSSTQ (SEQ ID NO:4), or AQQSY (SEQ ID NO:5) (FIG. 3B).

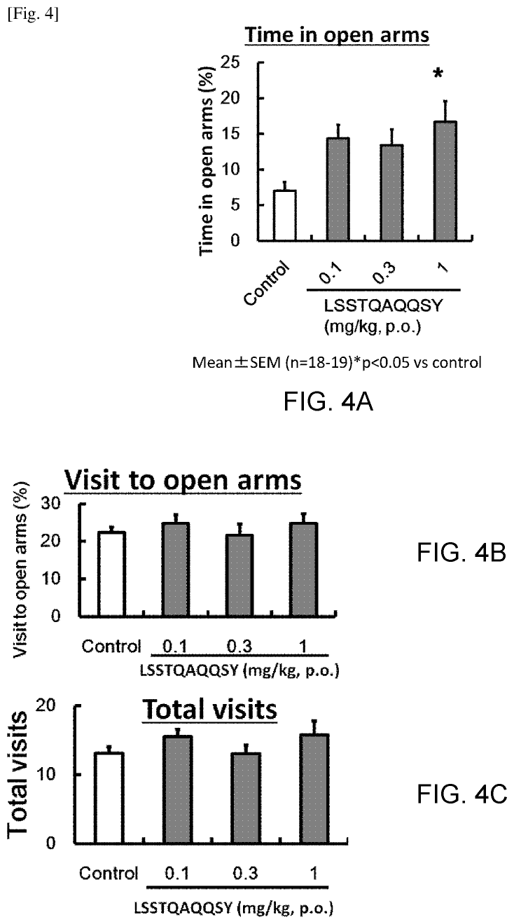

FIGS. 4A-C show the results of an elevated plus maze test in mice orally administered the peptide LSSTQAQQSY (SEQ ID NO:1). FIG. 4A shows the percentage of time the mice spent in the open arms of the maze. FIG. 4B shows the percentage of visits to arms of the maze which were made to the open arms. FIG. 4C shows the total number of visits to both the open and closed arms of the maze.

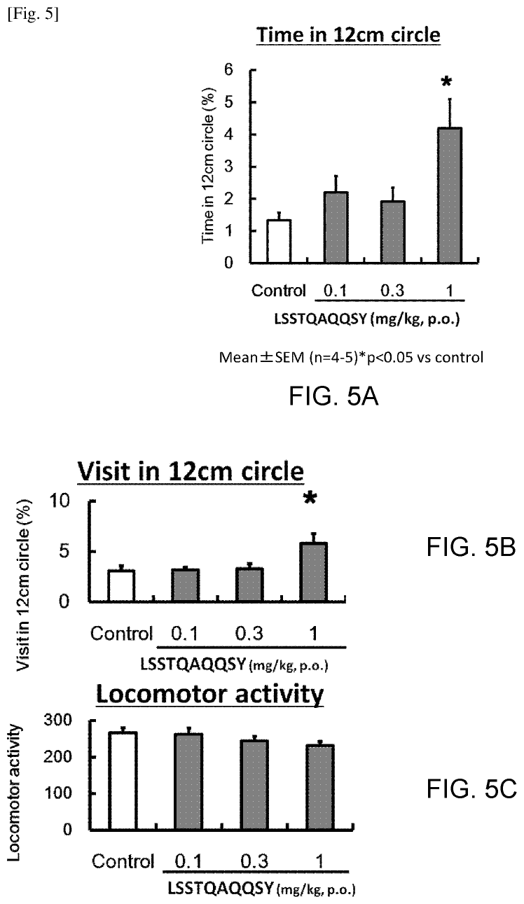

FIGS. 5A-5C show the results of an open field test in mice orally administered the peptide LSSTQAQQSY (SEQ ID NO:1). FIG. 5A shows the percentage of time the mice spent in the 12 cm circle at the center of the test chamber. FIG. 5B: shows the number of visits to the 12 cm circle. FIG. 5C shows the locomotor activity of the mice.

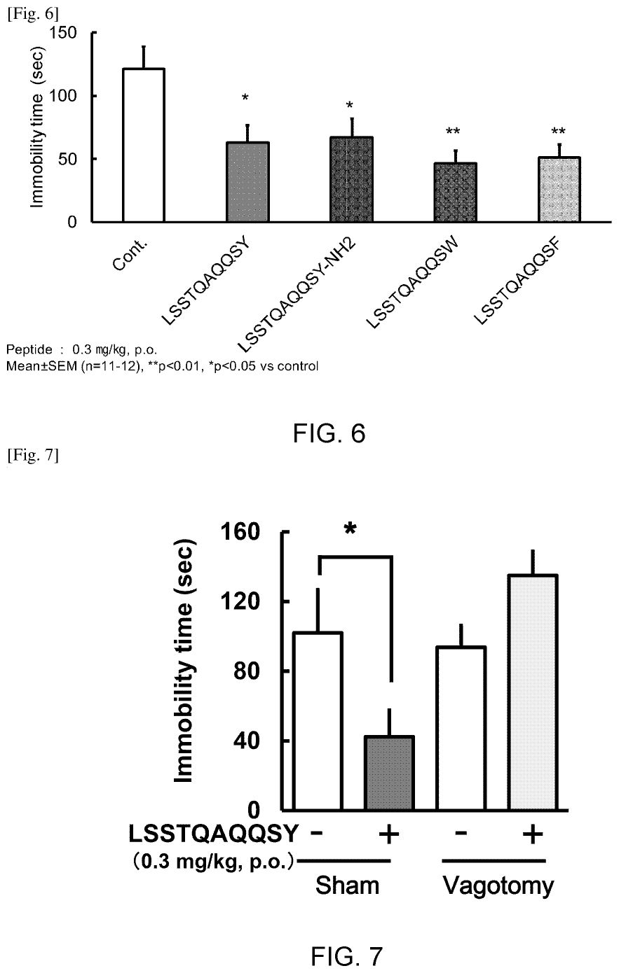

FIG. 6 shows the results of a tail suspension test in mice orally administered the peptide LSSTQAQQSY (SEQ ID NO:1), LSSTQAQQSW (SEQ ID NO:6), or LSSTQAQQSF (SEQ ID NO:7), or the C-terminal amidated peptide conjugate LSSTQAQQSY-NH₂ (SEQ ID NO:36).

FIG. 7 shows the results of a tail suspension test in vagotomized mice and sham operated mice orally administered the peptide LSSTQAQQSY (SEQ ID NO:1).

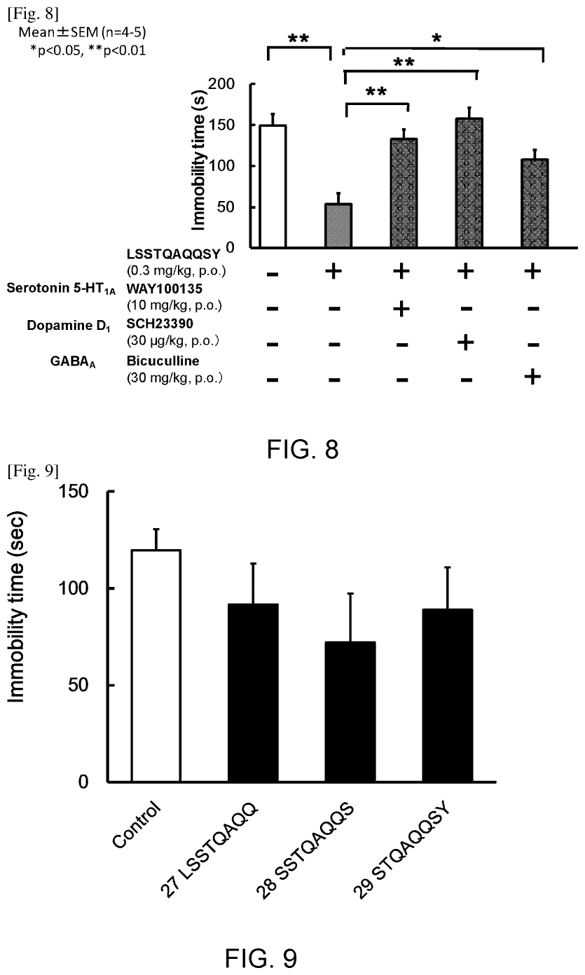

FIG. 8 shows the results of a tail suspension test in mice orally administered the peptide LSSTQAQQSY (SEQ ID NO:1) alone or in combination with an antagonist of serotonin 5-HT_{1A}, dopamine D₁, or GABA_{A} receptors.

FIG. 9 shows the results of a tail suspension test in mice orally administered the peptides LSSTQAQQ (SEQ ID NO:27), SSTQAQQS (SEQ ID NO:28), and STQAQQSY (SEQ ID NO:29).

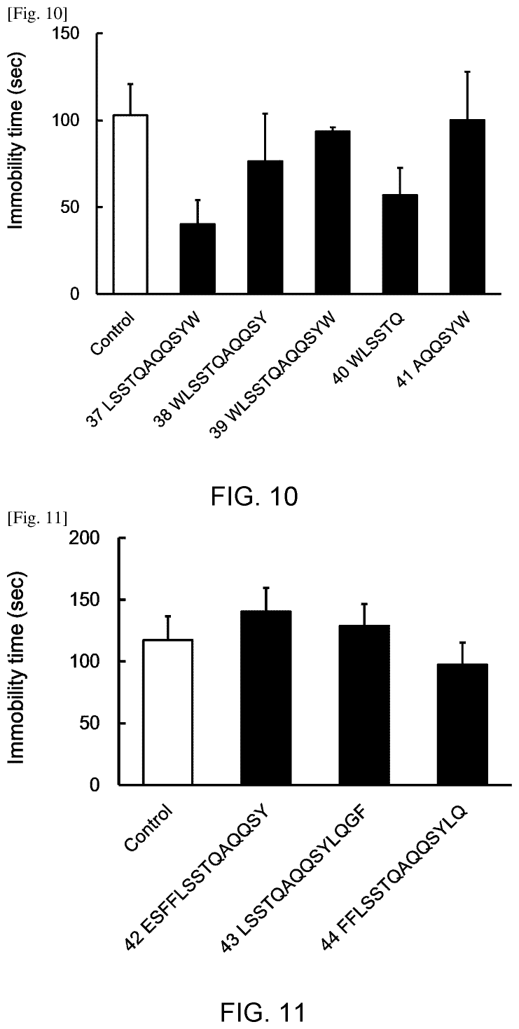

FIG. 10 shows the results of a tail suspension test in mice orally administered the peptides LSSTQAQQSYW (SEQ ID NO:37), WLSSTQAQQSY (SEQ ID NO:38), WLSSTQAQQSYW (SEQ ID NO:39), WLSSTQ (SEQ ID NO:40), and AQQSYW (SEQ ID NO:41).

FIG. 11 shows the results of a tail suspension test in mice orally administered the peptides ESFFLSSTQAQQSY (SEQ ID NO:42), LSSTQAQQSYLQGF (SEQ ID NO:43), and FFLSSTQAQQSYLQ (SEQ ID NO:44).

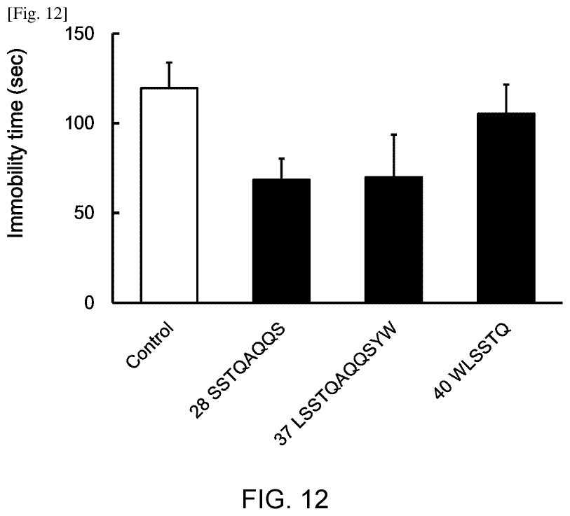

FIG. 12 shows the results of a tail suspension test in mice orally administered the peptides SSTQAQQS (SEQ ID NO:28), LSSTQAQQSYW (SEQ ID NO:37) and WLSSTQ (SEQ ID NO:40).

### Description of Embodiments

### 5. DETAILED DESCRIPTION

### 5.1. Peptides

The disclosure provides peptides and salts thereof that have an amino acid sequence that comprises or consists of at least 5 consecutive amino acids from the amino acid sequence LSSTQAQQSX₁ (SEQ ID NO:34), where X₁ is Y, W, or F, but not consisting of the amino acid sequence LSSTQAQQSY (SEQ ID NO:1), LSSTQAQQS (SEQ ID NO:2), SSTQAQQSY (SEQ ID NO:3), LSSTQ (SEQ ID NO:4), AQQSY (SEQ ID NO:5), LSSTQAQQSW (SEQ ID NO:6), or LSSTQAQQSF (SEQ ID NO:7). It should be understood that when an embodiment described herein refers to a “peptide,” the embodiment encompasses the peptide per se as well as salts of the peptide even though the embodiment may not explicitly recite the expression “or salt thereof” or similar, unless required otherwise by context.

The peptides of the disclosure are 5 to 15 amino acids (i.e., 5, 6, 7, 8, 9, 10, 11, 12, 13, 14 or 15 amino acids) in length. In some embodiments, a peptide of the disclosure is 5 amino acids in length. In other embodiments, a peptide of the disclosure is 6 amino acids in length. In other embodiments, a peptide of the disclosure is 7 amino acids in length. In other embodiments, a peptide of the disclosure is 8 amino acids in length. In other embodiments, a peptide of the disclosure is 9 amino acids in length. In other embodiments, a peptide of the disclosure is 10 amino acids in length. In other embodiments, a peptide of the disclosure is 11 amino acids in length. In other embodiments, a peptide of the disclosure is 12 amino acids in length. In other embodiments, a peptide of the disclosure is 13 amino acids in length. In other embodiments, a peptide of the disclosure is 14 amino acids in length. In other embodiments, a peptide of the disclosure is 15 amino acids in length.

A peptide of the disclosure generally comprises or consists of at least 5 consecutive amino acids from the amino acid sequence LSSTQAQQSX₁ (SEQ ID NO:34). Accordingly, in some embodiments, the peptide comprises or consists of the amino acid sequence SSTQA (SEQ ID NO:8). In other embodiments, the peptide comprises or consists of the amino acid sequence STQAQ (SEQ ID NO:9). In other embodiments, the peptide comprises or consists of the amino acid sequence TQAQQ (SEQ ID NO:10). In other embodiments, the peptide comprises or consists of the amino acid sequence QAQQS (SEQ ID NO: 11). In other embodiments, the peptide comprises or consists of the amino acid sequence AQQSW (SEQ ID NO:12). In other embodiments, the peptide comprises or consists of the amino acid sequence or AQQSF (SEQ ID NO:13). In other embodiments, the peptide comprises the amino acid sequence LSSTQ (SEQ ID NO:4). In other embodiments, the peptide comprises the amino acid sequence AQQSY (SEQ ID NO:5).

In certain aspects, a peptide of the disclosure comprises or consists of at least 6 consecutive amino acids from the amino acid sequence LSSTQAQQSX₁ (SEQ ID NO:34). Accordingly, in some embodiments, the peptide comprises or consists of the amino acid sequence LSSTQA (SEQ ID NO:14). In other embodiments, the peptide comprises or consists of the amino acid sequence SSTQAQ (SEQ ID NO:15). In other embodiments, the peptide comprises or consists of the amino acid sequence STQAQQ (SEQ ID NO:16). In other embodiments, the peptide comprises or consists of the amino acid sequence TQAQQS (SEQ ID NO:17). In other embodiments, the peptide comprises or consists of the amino acid sequence QAQQSY (SEQ ID NO:18). In other embodiments, the peptide comprises or consists of the amino acid sequence QAQQSW (SEQ ID NO:19). In other embodiments, the peptide comprises or consists of the amino acid sequence QAQQSF (SEQ ID NO:20).

In other aspects, a peptide of the disclosure comprises or consists of at least 7 consecutive amino acids from the amino acid sequence LSSTQAQQSX₁ (SEQ ID NO:34). Accordingly, in some embodiments, the peptide comprises or consists of the amino acid sequence LSSTQAQ (SEQ ID NO:21). In other embodiments, the peptide comprises or consists of the amino acid sequence SSTQAQQ (SEQ ID NO:22). In other embodiments, the peptide comprises or consists of the amino acid sequence STQAQQS (SEQ ID NO:23). In other embodiments, the peptide comprises or consists of the amino acid sequence TQAQQSY (SEQ ID NO:24). In other embodiments, the peptide comprises or consists of the amino acid sequence TQAQQSW (SEQ ID NO:25). In other embodiments, the peptide comprises or consists of the amino acid sequence TQAQQSF (SEQ ID NO:26).

In yet other aspects, a peptide of the disclosure comprises or consists of at least 8 consecutive amino acids from the amino acid sequence LSSTQAQQSX₁ (SEQ ID NO:34). Accordingly, in some embodiments, the peptide comprises or consists of the amino acid sequence LSSTQAQQ (SEQ ID NO:27). In other embodiments, the peptide comprises or consists of the amino acid sequence SSTQAQQS (SEQ ID NO:28). In other embodiments, the peptide comprises or consists of the amino acid sequence STQAQQSY (SEQ ID NO:29). In other embodiments, the peptide comprises or consists of the amino acid sequence STQAQQSW (SEQ ID NO:30). In other embodiments, the peptide comprises or consists of the amino acid sequence STQAQQSF (SEQ ID NO:31).

In yet further aspects, a peptide of the disclosure comprises or consists of at least 9 consecutive amino acids from the amino acid sequence LSSTQAQQSX₁ (SEQ ID NO:34). Accordingly, in some embodiments, the peptide comprises or consists of the amino acid sequence SSTQAQQSW (SEQ ID NO:32). In other embodiments, the peptide comprises or consists of the amino acid sequence SSTQAQQSF (SEQ ID NO:33). In other embodiments, the peptide comprises the amino acid sequence LSSTQAQQS (SEQ ID NO:2). In other embodiments, the peptide comprises the amino acid sequence SSTQAQQSY (SEQ ID NO:3).

In yet further aspects, a peptide of the disclosure comprises 10 consecutive amino acids from the amino acid sequence LSSTQAQQSX₁ (SEQ ID NO:34) and is 11, 12, 13, 14, or 15 amino acids in length. In some embodiments, the peptide comprises the amino acid sequence LSSTQAQQSY (SEQ ID NO:1). In other embodiments, the peptide comprises the amino acid sequence LSSTQAQQSW (SEQ ID NO:6). In other embodiments, the peptide comprises the amino acid sequence LSSTQAQQSF (SEQ ID NO:7).

Peptides that are 6 to 15 amino acids in lengths (i.e., peptides that are 6, 7, 8, 9, 10, 11, 12, 13, 14, or 15 amino acids long) can comprise, for example, an amino acid sequence identified in one of the previously described embodiments (i.e., comprise a sequence having at least 5, 6, 7, 8, 9, or 10 consecutive amino acids from SEQ ID NO:34) and one or more amino acids that naturally flank the sequence LSSTQAQQSY (SEQ ID NO:1) in the soybean storage protein β-conglycinin. For example, a peptide comprising the amino acid sequence LSSTQ (SEQ ID NO:4) and that is 6, 7, 8, 9, 10, 11, 12, 13, 14, or 15 amino acids in length can comprise an amino acid sequence N-terminal to the LSSTQ (SEQ ID NO:4) corresponding to the 1, 2, 3, 4, 5, 6, 7, 8, 9, or 10 amino acids that are N-terminal to LSSTQAQQSY (SEQ ID NO:1) in the sequence of β-conglycinin. As another example, a peptide comprising the amino acid sequence AQQSY (SEQ ID NO:5) and that is 6, 7, 8, 9, 10, 11, 12, 13, 14, or 15 amino acids in length can comprise an amino acid sequence C-terminal to the AQQSY (SEQ ID NO:5) corresponding to 1, 2, 3, 4, 5, 6, 7, 8, 9, or 10 amino acids that are C-terminal to LSSTQAQQSY (SEQ ID NO:1) in the sequence of β-conglycinin

SEQ ID NO:35 is a 30-mer peptide consisting of LSSTQAQQSX₁ (SEQ ID NO:34) flanked by 10 amino acids at each of the N- and C-termini that are present in the sequence of β-conglycinin. Thus, the first 10 amino acids in SEQ ID NO:35 represent the 10 amino acids N-terminal to LSSTQAQQSY (SEQ ID NO:1) in the sequence of β-conglycinin and the last 10 amino acids in SEQ ID NO:35 represent the 10 amino acids C-terminal to LSSTQAQQSY (SEQ ID NO:1) in the sequence of β-conglycinin.

In certain aspects, the sequence of a peptide of the disclosure is derived from SEQ ID NO:35. Accordingly, the present disclosure provides a peptide which is 5 to 15 amino acids in length and comprises or consists of 5 to 15 consecutive amino acids from SEQ ID NO:35, provided that the peptide comprises at least 5 consecutive amino acids from the amino acid sequence LSSTQAQQSX₁ (SEQ ID NO:34), and provided that the peptide is not LSSTQAQQSY (SEQ ID NO:1), LSSTQAQQS (SEQ ID NO:2), SSTQAQQSY (SEQ ID NO:3), LSSTQ (SEQ ID NO:4), AQQSY (SEQ ID NO:5), LSSTQAQQSW (SEQ ID NO:6), or LSSTQAQQSF (SEQ ID NO:7).

Peptides having sequence variations relative to SEQ ID NO:35 are also contemplated herein. Accordingly, in other embodiments, the peptide can have an amino acid sequence comprising one or more amino acid substitutions as compared to an amino acid sequence consisting of 5 to 15 consecutive amino acids from SEQ ID NO:35, provided that the peptide comprises at least 5 consecutive amino acids from the amino acid sequence LSSTQAQQSX₁ (SEQ ID NO:34) that have not been substituted. The maximum number of amino acid substitutions that a given peptide can have relative to a sequence consisting of 5 to 15 consecutive amino acids in SEQ ID NO:35 can vary depending on the length of the peptide, and can range from 1 amino acid substitution for a peptide that is 6 amino acids in length to 10 amino acid substitutions for a peptide that is 15 amino acids in length, provided that the peptide comprises at least 5 consecutive amino acids from the amino acid sequence LSSTQAQQSX₁ (SEQ ID NO:34) that have not been substituted. Permissible amino acid substitutions include, but are not limited to, substitution with conservative amino acids and/or amino acid analogs.

Preferably, a peptide of the disclosure having one or more amino acid substitutions as compared to the corresponding sequence in SEQ ID NO:35 has 1, 2, 3, 4, 5 or more conservative amino acid substitutions as compared to the corresponding amino acid in SEQ ID NO:35. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). In certain embodiments, all amino acid substitutions as compared to SEQ ID NO:35 are conservative.

In some embodiments, a peptide of the disclosure has an amino acid having an aromatic side chain (e.g., phenylalanine, tryptophan, or tyrosine) at the N-terminus and/or C-terminus of the peptide. In some embodiments, a peptide of the disclosure has an amino acid having an aromatic side chain at the C-terminus but not the N-terminus. In some embodiments, the peptide has a C-terminal tryptophan.

A peptide of the disclosure having amino acid substitutions as compared to the corresponding sequence in SEQ ID NO:35 can include one or more amino acid analogs (e.g., 1, 2, 3, 4, or 5 amino acid analogs). Generally, as used herein, an amino acid refers to a naturally occurring L-stereoisomer, and in some embodiments, each of the amino acids in the peptide is a naturally occurring L stereoisomer. An amino acid analog refers to a D-stereoisomer or an unnatural amino acid. For example, unnatural amino acids include, but are not limited to azetidinecarboxylic acid, 2-aminoadipic acid, 3-aminoadipic acid, β-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline.

The peptides can be entirely L-amino acids, entirely D-amino acids, or a mixture of L-amino acids and D-amino acids, with peptides that are entirely L-amino acids being preferred. Peptides containing two or more asymmetric carbon atoms can be any form of mixtures of enantiomers or diastereomers in any ratio.

The peptides can be in the form of salts, preferably comprising counterions that are pharmaceutically acceptable (e.g., chloride, sulfate, citrate, phosphate, acetate, sodium, potassium, calcium, magnesium). Peptide salts can be acid addition salts or a base addition salts. Exemplary acids that can be used to make an acid addition salt include hydrochloric acid, sulfuric acid, nitric acid, phosphoric acid, hydrobromic acid, perchloric acid, citric acid, succinic acid, maleic acid, fumaric acid, malic acid, tartaric acid, p-toluenesulfonic acid, benzenesulfonic acid, methanesulfonic acid, and trifluoroacetic acid. Exemplary bases that can be used to make a base addition salt include sodium hydroxide, potassium hydroxide, bases of alkali metals, such as lithium hydroxide, calcium hydroxide, and bases of alkaline earth metal salts such as magnesium hydroxide. Additional acids and bases that can be used to make pharmaceutically acceptable salts are described in Stahl and Wermuth, eds., 2008, Handbook of Pharmaceutical Salts: Properties, Selection and Use, Verlag Helvetica Chimica Acta, Zurich, Switzerland, the contents of which are incorporated herein by reference in their entireties.

The peptides be obtained, for example, by liquid phase peptide synthesis or solid phase peptide synthesis according to methods known in the art (e.g., as described in Benoiton, N., 2006, Chemistry of Peptide Synthesis, CRC Press, Boca Raton, Fla.; Howl, J., ed., 2005, Peptide Synthesis and Applications, Humana Press, Totowa, N.J.; Chan and White, eds., 2000, Fmoc Solid Phase Synthesis: A Practical Approach, Oxford University Press, Oxford, UK). Custom peptide synthesis is also available commercially from numerous vendors (e.g., ABI Scientific (Sterling, Va.); AnaSpec (Freemont Calif.); Pepscan, (Lelystad, Netherlands), Neo Scientific (Cambridge, Mass.); Sigma-Aldrich (St. Louis, Mo.)).

### 5.2. Peptide Conjugates

The disclosure provides peptide conjugates and salts thereof that comprise a peptide moiety and a conjugate moiety. Attachment of a conjugate moiety to a peptide can provide, for example, improved water solubility, improved stability, and reduced clearance as compared to the non-conjugated peptide (Hamley, 2014, Biomacromolecules 15:1543-1559). Thus, peptide conjugates can in some instances be more suitable as therapeutic agents compared to their unconjugated counterparts. Exemplary peptide moieties are described in Section 5.2.1 and exemplary conjugate moieties are described in Section 5.2.2. It should be understood that when an embodiment described herein refers to a “peptide conjugate,” the embodiment encompasses the peptide conjugate per se as well as salts of the peptide conjugate even though the embodiment may not explicitly recite the expression “or salt thereof” or similar, unless required otherwise by context. Exemplary salts are described in Section 5.2.3.

### 5.2.1. Peptide Moieties

Peptide moieties have an amino acid sequence that comprises or consists of at least 5 consecutive amino acids from the amino acid sequence LSSTQAQQSX₁ (SEQ ID NO:34), but not consist of LSSTQAQQSY (SEQ ID NO:1), LSSTQAQQSW (SEQ ID NO:6), or LSSTQAQQSF (SEQ ID NO:7). The peptide moiety is attached (i.e., covalently) to one or more conjugate moieties (e.g., 1, 2, 3, 4, or 5 conjugate moieties).

The peptide moieties of the disclosure are 5 to 15 amino acids (i.e., 5, 6, 7, 8, 9, 10, 11, 12, 13, 14 or 15 amino acids) in length. In some embodiments, a peptide moiety of the disclosure is 5 amino acids in length. In other embodiments, a peptide moiety of the disclosure is 6 amino acids in length. In other embodiments, a peptide moiety of the disclosure is 7 amino acids in length. In other embodiments, a peptide moiety of the disclosure is 8 amino acids in length. In other embodiments, a peptide moiety of the disclosure is 9 amino acids in length. In other embodiments, a peptide moiety of the disclosure is 10 amino acids in length. In other embodiments, a peptide moiety of the disclosure is 11 amino acids in length. In other embodiments, a peptide moiety of the disclosure is 12 amino acids in length. In other embodiments, a peptide moiety of the disclosure is 13 amino acids in length. In other embodiments, a peptide moiety of the disclosure is 14 amino acids in length. In other embodiments, a peptide moiety of the disclosure is 15 amino acids in length.

A peptide moiety of the disclosure generally comprises or consists of at least 5 consecutive amino acids from the amino acid sequence LSSTQAQQSX₁ (SEQ ID NO:34). Accordingly, in some embodiments, the peptide moiety comprises or consists of the amino acid sequence LSSTQ (SEQ ID NO:4). In other embodiments, the peptide moiety comprises or consists of the amino acid sequence SSTQA (SEQ ID NO:8). In other embodiments, the peptide moiety comprises or consists of the amino acid sequence STQAQ (SEQ ID NO:9). In other embodiments, the peptide moiety comprises or consists of the amino acid sequence TQAQQ (SEQ ID NO:10). In other embodiments, the peptide moiety comprises or consists of the amino acid sequence QAQQS (SEQ ID NO: 11). In other embodiments, the peptide moiety comprises or consists of the amino acid sequence AQQSY (SEQ ID NO:5). In other embodiments, the peptide moiety comprises or consists of the amino acid sequence AQQSW (SEQ ID NO:12). In other embodiments, the peptide moiety comprises or consists of the amino acid sequence AQQSF (SEQ ID NO:13).

In certain aspects, a peptide moiety of the disclosure comprises or consists of at least 6 consecutive amino acids from the amino acid sequence LSSTQAQQSX₁ (SEQ ID NO:34). Accordingly, in some embodiments, the peptide moiety comprises or consists of the amino acid sequence LSSTQA (SEQ ID NO:14). In other embodiments, the peptide moiety comprises or consists of the amino acid sequence SSTQAQ (SEQ ID NO:15). In other embodiments, the peptide moiety comprises or consists of the amino acid sequence STQAQQ (SEQ ID NO:16). In other embodiments, the peptide moiety comprises or consists of the amino acid sequence TQAQQS (SEQ ID NO:17). In other embodiments, the peptide moiety comprises or consists of the amino acid sequence QAQQSY (SEQ ID NO:18). In other embodiments, the peptide moiety comprises or consists of the amino acid sequence QAQQSW (SEQ ID NO:19). In other embodiments, the peptide moiety comprises or consists of the amino acid sequence QAQQSF (SEQ ID NO:20).

In other aspects, a peptide moiety of the disclosure comprises or consists of at least 7 consecutive amino acids from the amino acid sequence LSSTQAQQSX₁ (SEQ ID NO:34). Accordingly, in some embodiments the peptide moiety comprises or consist of the amino acid sequence LSSTQAQ (SEQ ID NO:21). In other embodiments, the peptide moiety comprises or consists of the amino acid sequence SSTQAQQ (SEQ ID NO:22). In other embodiments, the peptide moiety comprises or consists of the amino acid sequence STQAQQS (SEQ ID NO:23). In other embodiments, the peptide moiety comprises or consists of the amino acid sequence TQAQQSY (SEQ ID NO:24). In other embodiments, the peptide moiety comprises or consists of the amino acid sequence TQAQQSW (SEQ ID NO:25). In other embodiments, the peptide moiety comprises or consists of the amino acid sequence TQAQQSF (SEQ ID NO:26).

In yet other aspects, a peptide moiety of the disclosure comprises or consists of at least 8 consecutive amino acids from the amino acid sequence LSSTQAQQSX₁ (SEQ ID NO:34). Accordingly, in some embodiments, the peptide moiety comprises or consists of the amino acid sequence LSSTQAQQ (SEQ ID NO:27). In other embodiments, the peptide moiety comprises or consists of the amino acid sequence SSTQAQQS (SEQ ID NO:28). In other embodiments, the peptide moiety comprises or consists of the amino acid sequence STQAQQSY (SEQ ID NO:29). In other embodiments, the peptide moiety comprises or consists of the amino acid sequence STQAQQSW (SEQ ID NO:30). In other embodiments, the peptide moiety comprises or consists of the amino acid sequence STQAQQSF (SEQ ID NO:31).

In yet further aspects, a peptide moiety of the disclosure comprises or consists of at least 9 consecutive amino acids from the amino acid sequence LSSTQAQQSX₁ (SEQ ID NO:34). Accordingly, in some embodiments, the peptide moiety comprises or consists of the amino acid sequence LSSTQAQQS (SEQ ID NO:2). In other embodiments, the peptide moiety comprises or consists of the amino acid sequence SSTQAQQSY (SEQ ID NO:3). In other embodiments, the peptide moiety comprises or consists of the amino acid sequence SSTQAQQSW (SEQ ID NO:32). In other embodiments, the peptide moiety comprises or consists of the amino acid sequence SSTQAQQSF (SEQ ID NO:33).

In yet further aspects, a peptide moiety of the disclosure comprises 10 consecutive amino acids from the amino acid sequence LSSTQAQQSX₁ (SEQ ID NO:34). In some embodiments, the peptide moiety comprises the amino acid sequence LSSTQAQQSY (SEQ ID NO:1). In other embodiments, the peptide moiety comprises the amino acid sequence LSSTQAQQSW (SEQ ID NO:6). In other embodiments, the peptide moiety comprises the amino acid sequence LSSTQAQQSF (SEQ ID NO:7).

Peptide moieties that are 6 to 15 amino acids in lengths (i.e., peptide moieties that are 6, 7, 8, 9, 10, 11, 12, 13, 14, or 15 amino acids long) can comprise, for example, an amino acid sequence identified in one of the previously described embodiments (i.e., comprise a sequence having at least 5, 6, 7, 8, 9, or 10 consecutive amino acids from SEQ ID NO:34) and one or more amino acids that naturally flank the sequence LSSTQAQQSY (SEQ ID NO:1) in the soybean storage protein β-conglycinin. For example, a peptide moiety comprising the amino acid sequence LSSTQ (SEQ ID NO:4) and that is 6, 7, 8, 9, 10, 11, 12, 13, 14, or 15 amino acids in length can comprise an amino acid sequence N-terminal to the LSSTQ (SEQ ID NO:4) corresponding to the 1, 2, 3, 4, 5, 6, 7, 8, 9, or 10 amino acids that are N-terminal to LSSTQAQQSY (SEQ ID NO:1) in the sequence of β-conglycinin. As another example, a peptide moiety comprising the amino acid sequence AQQSY (SEQ ID NO:5) and that is 6, 7, 8, 9, 10, 11, 12, 13, 14, or 15 amino acids in length can comprise an amino acid sequence C-terminal to the AQQSY (SEQ ID NO:5) corresponding to 1, 2, 3, 4, 5, 6, 7, 8, 9, or 10 amino acids that are C-terminal to LSSTQAQQSY (SEQ ID NO:1) in the sequence of β-conglycinin.

SEQ ID NO:35 is a 30-mer peptide consisting of LSSTQAQQSX₁ (SEQ ID NO:34) flanked by 10 amino acids at each of the N- and C-termini that are present in the sequence of β-conglycinin. Thus, the first 10 amino acids in SEQ ID NO:35 represent the 10 amino acids N-terminal to LSSTQAQQSY (SEQ ID NO:1) in the sequence of β-conglycinin and the last 10 amino acids in SEQ ID NO:35 represent the 10 amino acids C-terminal to LSSTQAQQSY (SEQ ID NO:1) in the sequence of β-conglycinin.

In certain aspects, the sequence of a peptide moiety of the disclosure is derived from SEQ ID NO:35. Accordingly, the present disclosure provides a peptide moiety which is 5 to 15 amino acids in length and comprises or consists of 5 to 15 amino acids in length and comprises or consists of 5 to 15 consecutive amino acids from SEQ ID NO:35, provided that the peptide moiety comprises at least 5 consecutive amino acids from the amino acid sequence LSSTQAQQSX₁ (SEQ ID NO:34), and provided that the peptide moiety is not LSSTQAQQSY (SEQ ID NO:1), LSSTQAQQSW (SEQ ID NO:6), or LSSTQAQQSF (SEQ ID NO:7).

Peptide moieties having sequence variations relative to SEQ ID NO:35 are also contemplated herein. Accordingly, in other embodiments, the peptide moiety can have an amino acid sequence comprising one or more amino acid substitutions as compared to an amino acid sequence consisting of 5 to 15 consecutive amino acids from SEQ ID NO:35, provided that the peptide moiety comprises at least 5 consecutive amino acids from the amino acid sequence LSSTQAQQSX₁ (SEQ ID NO:34) that have not been substituted. The maximum number of conservative amino acid substitutions that a given peptide moiety can have relative to a sequence consisting of 5 to 15 consecutive amino acids in SEQ ID NO:35 can vary depending on the length of the peptide moiety, and can range from 1 amino acid substitution for a peptide moiety that is 6 amino acids in length to 10 amino acid substitutions for a peptide moiety that is 15 amino acids in length, provided that the peptide moiety comprises at least 5 consecutive amino acids from the amino acid sequence LSSTQAQQSX₁ (SEQ ID NO:34) that have not been substituted. Permissible amino acid substitutions include, but are not limited to, substitution with conservative amino acids and/or amino acid analogs.

Preferably, a peptide moiety of the disclosure having one or more amino acid substitutions as compared to the corresponding sequence in SEQ ID NO:35 has 1, 2, 3, 4, 5 or more conservative amino acid substitutions as compared to the corresponding amino acid in SEQ ID NO:35. In certain embodiments, all amino acid substitutions as compared to SEQ ID NO:35 are conservative.

In some embodiments, a peptide moiety of the disclosure has an amino acid having an aromatic side chain (e.g., phenylalanine, tryptophan, or tyrosine) at the N-terminus and/or C-terminus of the peptide. In some embodiments, a peptide moiety of the disclosure has an amino acid having an aromatic side chain at the C-terminus but not the N-terminus. In some embodiments, the peptide moiety has a C-terminal tryptophan.

A peptide moiety of the disclosure having amino acid substitutions as compared to the corresponding sequence in SEQ ID NO:35 can include one or more amino acid analogs (e.g., 1, 2, 3, 4, or 5 amino acid analogs), for example as described in Section 5.1.

The peptide moieties can be entirely L-amino acids, entirely D-amino acids, or a mixture of L-amino acids and D-amino acids, with peptide moieties that are entirely L-amino acids being preferred. Peptide moieties containing two or more asymmetric carbon atoms can be any form of mixtures of enantiomers or diastereomers in any ratio.

Peptide moieties can be obtained, for example, by liquid phase peptide synthesis or solid phase peptide synthesis according to methods known in the art (e.g., as described in Benoiton, N., 2006, Chemistry of Peptide Synthesis, CRC Press, Boca Raton, Fla.; Howl, J., ed., 2005, Peptide Synthesis and Applications, Humana Press, Totowa, N.J.; Chan and White, eds., 2000, Fmoc Solid Phase Synthesis: A Practical Approach, Oxford University Press, Oxford, UK). Custom peptide synthesis is also available commercially from numerous vendors (e.g., ABI Scientific (Sterling, Va.); AnaSpec (Freemont Calif.); Pepscan, (Lelystad, Netherlands), Neo Scientific (Cambridge, Mass.); Sigma-Aldrich (St. Louis, Mo.)).

### 5.2.2. Conjugate Moieties

The peptide conjugates comprise one or more conjugate moieties (e.g., 1, 2, 3, 4, or 5 conjugate moieties) attached to the peptide moiety. The conjugate moiety or moieties can be attached to an N-terminal amino acid, a C-terminal amino acid, an amino acid that is neither an N-terminal amino acid or a C-terminal amino acid, or a combination thereof. For example, a peptide conjugate can comprise one conjugate moiety, preferably which is either attached to the N-terminal amino acid of the peptide moiety or attached to the C-terminal amino acid of the peptide moiety. As another example, a peptide conjugate can comprise two conjugate moieties, one of which is preferably attached to the N-terminal amino acid of the peptide moiety and the other of which is preferably attached to the C-terminal amino acid of the peptide moiety.

In embodiments in which the peptide conjugate comprises multiple conjugate moieties, each of the conjugate moieties can be the same, some of the conjugate moieties can be the same and others can be different, or all of the conjugate moieties can be different. For example, a peptide conjugate having two conjugate moieties can have two of the same conjugate moiety. Alternatively, a peptide conjugate having two conjugate moieties can have two different conjugate moieties. As another example, a peptide conjugate having three conjugate moieties can have three of the same conjugate moiety, three different conjugate moieties, or two of the same conjugate moiety and one different conjugate moiety.

A conjugate moiety can be attached to a peptide moiety, for example, at one of the peptide moiety's amino acid side chains, its backbone, its N-terminal amino group, or its C-terminal carboxylic acid group. For example, a conjugate moiety can be attached to an amino acid side chain to form a chemically modified amino acid, such as methionine sulfoxide, methionine sulfone, S-(carboxymethyl)-cysteine, S-(carboxymethyl)-cysteine sulfoxide and S-(carboxymethyl)-cysteine sulfone. Other side chain modifications include acylation of lysine ε-amino groups, N-alkylation of arginine, histidine, or lysine, and alkylation of glutamic or aspartic carboxylic acid groups. Conjugate moieties can be attached to the peptide backbone, for example to a nitrogen atom in the backbone (e.g., a methyl conjugate moiety can be introduced into a peptide conjugate's backbone by using an N-methyl amino acid to synthesize the peptide). Conjugate moieties can be attached to the N-terminal amino group of the peptide moiety to provide, for example, an N-terminus having an N-lower alkyl, N-di-lower alkyl, or N-acyl modifications. Conjugate moieties can be attached to the C-terminal carboxy group to provide, for example, a peptide conjugate having an amide, a lower alkyl amide, a dialkyl amide, or a lower alkyl ester at the C-terminus of the conjugate. A lower alkyl refers to a C₁-C₄ alkyl.

Exemplary conjugate moieties that can be used in the peptide conjugates include polymers, amine groups (e.g., amino (—NH₂), alkyl amino and dialkyl amino), acyls groups (e.g., formyl or acetyl), alkyl groups (e.g., C₁-C₄ alkyl), phosphate groups, lipids and sugars.

In some embodiments, at least one, more than one, or all of the conjugate moieties in the peptide conjugate comprise(s) a polymer. Exemplary polymers that can be used as conjugate moieties include polyethylene glycol, polyvinyl pyrrolidone, polylactic-co-glycolic acid, N-(2-hydroxypropyl) methacrylamide copolymer, polyglutamic acid, and polysaccharides. In some embodiments, at least one, more than one, or all of the conjugate moieties in the peptide conjugate comprise(s) a polyethylene glycol. In some embodiments, at least one, more than one, or all of the conjugate moieties in the peptide conjugate comprise(s) polyvinyl pyrrolidone. In some embodiments, at least one, more than one, or all of the conjugate moieties in the peptide conjugate comprise(s) polylactic-co-glycolic acid. In some embodiments, at least one, more than one, or all of the conjugate moieties in the peptide conjugate comprise(s) N-(2-hydroxypropyl) methacrylamide copolymer. In some embodiments, at least one, more than one, or all of the conjugate moieties in the peptide conjugate comprise(s) polyglutamic acid. In some embodiments, at least one, more than one, or all of the conjugate moieties in the peptide conjugate comprise(s) comprises a polysaccharide.

In some embodiments, at least one, more than one, or all of the conjugate moieties in the peptide conjugate comprise(s) an amine group. Exemplary amine groups include amino (—NH₂), alkyl amino, and dialkyl amino groups. The alkyl groups can be, for example, a C₁-C₄ alkyl. In some embodiments, at least one, more than one, or all of the conjugate moieties in the peptide conjugate comprise(s) an amino group. In some embodiments, at least one, more than one, or all of the conjugate moieties in the peptide conjugate comprise(s) an alkyl amino group. In some embodiments, at least one, more than one, or all of the conjugate moieties in the peptide conjugate comprise(s) a dialkyl amino group.

In some embodiments, at least one, more than one, or all of the conjugate moieties in the peptide conjugate comprise(s) an acyl group. Exemplary acyl groups include formyl groups and acetyl groups. In some embodiments, at least one, more than one, or all of the conjugate moieties in the peptide conjugate comprise(s) a formyl group. In some embodiments, at least one, more than one, or all of the conjugate moieties in the peptide conjugate comprise(s) an acetyl group.

In some embodiments, at least one, more than one, or all of the conjugate moieties in the peptide conjugate comprise(s) an alkyl group. In exemplary embodiments, the alkyl group is a lower alkyl group, such as methyl or ethyl. In some embodiments, at least one, more than one, or all of the conjugate moieties in the peptide conjugate comprise(s) a methyl group. In some embodiments, at least one, more than one, or all of the conjugate moieties in the peptide conjugate comprise(s) an ethyl group.

In some embodiments, at least one, more than one, or all of the conjugate moieties in the peptide conjugate comprise(s) a phosphate group, for example attached to the side chain of a serine, threonine, or tyrosine.

In some embodiments, at least one, more than one, or all of the conjugate moieties in the peptide conjugate comprise(s) a lipid.

In some embodiments, at least one, more than one, or all of the conjugate moieties in the peptide conjugate comprise(s) a sugar.

In some embodiments, the peptide conjugate comprises one or more of the peptide modifications described in PCT international application no. PCT/JP2016/056453, the contents of which are incorporated herein by reference in their entireties.

Processes for attaching conjugate moieties to peptide moieties are known in the art and can be used to obtain the peptide conjugates described herein (e.g., as described in Basle et al., 2010, Chemistry & Biology 17:213-227; Benoiton, N., 2006, Chemistry of Peptide Synthesis, CRC Press, Boca Raton, Fla.; Ernst and Leumann, eds., 1995, Modern Synthetic Methods, Verlag Helvetica Chimica Acta, Basel, Switzerland; Hamley, 2014, Biomacromolecules 15:1543-1559; Lundblad, R., 1995, Techniques in Protein Modification, CRC Press, Boca Raton, Fla.). Custom synthesis of peptide conjugates is also commercially available from numerous vendors (e.g., ABI Scientific (Sterling, Va.); AnaSpec (Freemont Calif.); Pepscan, (Lelystad, Netherlands), Neo Scientific (Cambridge, Mass.); Sigma-Aldrich (St. Louis, Mo.), variously offering, for example, peptides having N-terminal conjugate moieties such as an acetyl group, a formyl group, a fatty acid, and alkyl amino groups, peptides having C-terminal conjugate moieties such as an amido group, alkyl amino groups, and alkyl groups, peptides conjugated to fatty acids, peptides conjugated to polyethylene glycol, and peptides having a phosphate conjugate moiety (e.g., comprising phosphoserine, phosphothreonine, or phosphotyrosine)).

### 5.2.3. Salts of Peptide Conjugates

The peptide conjugates can be in the form of salts, preferably comprising counterions that are pharmaceutically acceptable (e.g., chloride, sulfate, citrate, phosphate, acetate, sodium, potassium, calcium, magnesium). The peptide conjugate salts can be an acid addition salt or a base addition salt. Exemplary acids that can be used to make an acid addition salt include hydrochloric acid, sulfuric acid, nitric acid, phosphoric acid, hydrobromic acid, perchloric acid, citric acid, succinic acid, maleic acid, fumaric acid, malic acid, tartaric acid, p-toluenesulfonic acid, benzenesulfonic acid, methanesulfonic acid, and trifluoroacetic acid. Exemplary bases that can be used to make a base addition salt include sodium hydroxide, potassium hydroxide, bases of alkali metals, such as lithium hydroxide, calcium hydroxide, and bases of alkaline earth metal salts such as magnesium hydroxide. Additional acids and bases that can be used to make pharmaceutically acceptable salts are described in Stahl and Wermuth, eds., 2008, Handbook of Pharmaceutical Salts: Properties, Selection and Use, Verlag Helvetica Chimica Acta, Zurich, Switzerland, the contents of which are incorporated herein by reference in their entireties.

### 5.3. Pharmaceutical Compositions

The peptides, salts thereof, peptide conjugates, and salts thereof described in Sections 5.1 and 5.2 can be formulated into pharmaceutical compositions comprising one or more pharmaceutically acceptable carriers, diluents, and/or excipients.

The pharmaceutical compositions can be formulated for topical, enteral (e.g., oral or rectal) or parenteral (e.g., intramuscular or intravenous) administration. Preferably, the pharmaceutical compositions are formulated for oral administration. The pharmaceutical compositions can be formulated as any suitable dosage form, such as a tablet, a capsule, granules, a powder, a syrup, a suspension, a suppository, an ointment, a cream, a gel, a patch, an inhalation, a solution for injection and the like according to techniques known in the art (e.g., as described in Allen et al., eds., 2012, Remington: The Science and Practice of Pharmacy, 22^{nd} Edition, Pharmaceutical Press, London, UK).

Liquid pharmaceutical compositions can comprise a suitable solvent such as water, saline, a glucose solution, or ethanol. Liquid pharmaceutical composition can be formulated, for example, by dissolving or suspending an amount of a compound of the disclosure in the solvent. Buffering agents, preservatives, flavoring agents and colorants can each optionally be included in liquid formulations. Solid pharmaceutical compositions can include binders, lubricants, disintegrating agents, coloring agents, flavoring agents, flow-inducing agents, and melting agents. For example, solid formulations can contain an inert carrier such as lactose, gelatin, agar, starch, sucrose, glucose, methyl cellulose, dicalcium phosphate, calcium sulfate, mannitol, sorbitol, crystalline cellulose and the like. Binders include starch, gelatin, sugars such as glucose, lactose and trehalose, corn starch, calcium lactate, natural and synthetic gums such as acacia, tragacanth, or sodium alginate, povidone, carboxymethylcellulose, hydroxypropyl cellulose, polyethylene glycol, waxes, and the like. Lubricants include, for example, sodium oleate, sodium stearate, magnesium stearate, stearic acid, sodium stearyl fumarate, anhydrous silicic acid, talc and the like. Disintegrators include, for example, starch, methyl cellulose, agar, bentonite, xanthan gum, croscarmellose sodium, sodium starch glycolate and the like. Solid pharmaceutical compositions can be enteric coated, for example, with methyl methacrylate polymer, ethyl cellulose, or carnauba wax. Additional agents that can be included in the pharmaceutical compositions are described in PCT international application no. PCT/JP2016/056453, the contents of which are incorporated herein by reference in their entireties.

The compounds of the disclosure can be included in pharmaceutical compositions in any suitable amount. Typically, a compound of the disclosure constitutes 0.1 wt % to less than 100 wt % of the pharmaceutical composition (e.g., 1 wt % to 99 wt %, 1 wt % to 90 wt %, 5 wt % to 80 wt %, 10 wt % to 75 wt %, or 15 wt % to 50 wt % of the pharmaceutical composition, or any wt % range bound by any two of the foregoing values).

### 5.4. Food Products

The peptides, salts thereof, peptide conjugates, and salts thereof described in Sections 5.1 and 5.2 can be formulated into food products such as a dietary supplement or a functional food.

The dietary supplement can be, for example, in the form of a tablet, a capsule, a softgel, a gelcap, a liquid, or a powder. Vitamins, minerals, herbs or other botanicals, amino acids, proteins, fiber, fatty acids, or a combination thereof can be included in the supplement.

Functional foods are foods in which a compound of the disclosure has been added or incorporated. For example, the functional food can be a drink such as coffee, cocoa, juice, a soft drink, a mineral beverage, a tea beverage, green tea, black tea, oolong tea, a milk drink, a lactic acid bacteria drink, a yogurt drink, a carbonated beverage, a non-alcoholic beverage, or an alcoholic beverage. The functional food can be a confectionery (e.g., a hard candy, a gum, a gummy, a jelly, a pudding, a mousse, a cake, a candy, a cookie, a cracker, a biscuit, a chocolate, a frozen dessert such as ice cream, ice candy, sherbet, shaved ice and the like), a dressing, a seasoning, a soy processed food (e.g., tofu, miso, soy sauce, bean curd, soybean flour, natto, etc.), a meat processed food (e.g., hamburger, meatloaf, meatballs, etc.), a fish meat processed food (e.g., boiled fish paste, such as fish sausage), a jelly-like food (e.g., jelly, agar, a jelly-like beverage, etc.), and the like.

### 5.5. Methods of Treatment

The peptides, salts thereof, peptide conjugates, and salts thereof described in Sections 5.1 and 5.2, the pharmaceutical compositions described in Section 5.3, and the food products described in Section 5.4, can be used to treat or prevent a mood disorder. Mood disorders that can be treated or prevented include depression, bipolar disorder, and adjustment disorder.

The disclosure provides a method for treating a subject suffering from a mood disorder comprising administering to the subject a compound of the disclosure or a pharmaceutical composition comprising a compound of the disclosure in an amount effective to treat the subject. The disclosure also provides a method for treating or preventing a mood disorder comprising administering an amount of a food product comprising a compound of the disclosure to a subject prone to or suffering from a mood disorder. In some embodiments, the subject is prone to suffer from a mood disorder. In other embodiments, the subject is suffering from a mood disorder.

In some embodiments of the methods described in the preceding paragraph, the mood disorder comprises depression. In some embodiments of the methods described in the preceding paragraph, the mood disorder comprises bipolar disorder. In some embodiments of the methods described in the preceding paragraph, the mood disorder comprises adjustment disorder.

The peptides, salts thereof, peptide conjugates, and salts thereof described in Sections 5.1 and 5.2, the pharmaceutical compositions described in Section 5.3, and the food products described in Section 5.4, can be used to treat or prevent an anxiety disorder.

The disclosure provides a method for treating a subject suffering from an anxiety disorder comprising administering to the subject a compound of the disclosure or a pharmaceutical composition comprising a compound of the disclosure in an amount effective to treat the subject. The disclosure also provides a method for treating or preventing an anxiety disorder comprising administering an amount of a food product comprising a compound of the disclosure to a subject prone to or suffering from the anxiety disorder. In some embodiments, the subject is prone to suffer from an anxiety disorder. In other embodiments, the subject is suffering from an anxiety disorder.

The peptides, salts thereof, peptide conjugates, and salts thereof described in Sections 5.1 and 5.2, the pharmaceutical compositions described in Section 5.3, and the food products described in Section 5.4, can be used to treat or prevent disorder of diminished motivation. Disorders of diminished motivation that can be treated or prevented include apathy, abulia, and akinetic mutism.

The disclosure provides a method for treating a subject suffering from a disorder of diminished motivation comprising administering to the subject a compound of the disclosure in an amount effective to treat the subject. The disclosure also provides a method for treating or preventing a disorder of diminished motivation comprising administering an amount of a food product comprising a compound of the disclosure to a subject prone to or suffering from a disorder of diminished motivation. In some embodiments, the subject is prone to suffer from a disorder of diminished motivation. In other embodiments, the subject is suffering from a disorder of diminished motivation.

In some embodiments of the methods described in the preceding paragraph, the disorder of diminished motivation comprises apathy. In other embodiments of the methods described in the preceding paragraph, the disorder of diminished motivation comprises abulia. In other embodiments of the methods described in the preceding paragraph, the disorder of diminished motivation comprises akinetic mutism.

The subjects of the methods described herein are preferably mammals, e.g., humans or domestic pets (e.g., cat, dog). Subjects can be of any age, but are preferably adults (e.g., a human subject who is 18 years old or more, 25 years old or more, 35 years old or more, 45 years old or more, 55 years old or more, etc.). In some embodiments, the subject is elderly (e.g., a human subject who is 65 years old or more, 70 years old or more, 75 years old or more, or 80 years old or more).

The compounds of the disclosure and pharmaceutical compositions containing them can be administered orally, topically, rectally, or parenterally, with oral administration being preferred. The method of administration can vary depending on the condition and age of the subject.

Appropriate daily dosages of the compounds of the disclosure can range from 0.005 mg/kg to 500 mg/kg of body weight per day (e.g., 0.005 mg/kg to 100 mg/kg, 0.005 mg/kg to 30 mg/kg, 0.005 mg/kg to 1 mg/kg, 0.01 mg/kg to 30 mg/kg, 0.01 mg/kg to 3 mg/kg, 0.01 mg/kg to 1 mg/kg, 0.02 mg/kg to 5 mg/kg, 0.02 mg/kg to 2 mg/kg, 0.02 mg/kg to 1 mg/kg). Alternatively, the compounds can be administered at a fixed dose ranging from 0.1 mg to 50 g per day (e.g., 0.1 mg to 10 g, 0.1 mg to 3 g, 0.1 mg to 100 mg, 0.1 mg to 1 mg, 0.3 mg to 3 g, or 0.3 mg to 100 mg). For administration of a pharmaceutical composition containing a compound of the disclosure, an amount of the pharmaceutical composition can be administered that contains an amount of the compound that is within one of the foregoing ranges. Likewise, for administration of one or more food products containing the compound, an amount of one or more food products can be administered that contain an amount of the compound that is within one of the foregoing ranges.

### 6. EXAMPLES

### 6.1. Methods

### 6.1.1. Tail Suspension Test

The tail suspension test is an experimental method used to screen potential antidepressant drugs (Can et al., 2012, J Vis Exp., 59:e3769). In the test as implemented in the Examples, a test substance is orally administered to mice (ddY mice, males, 24˜30 g) and 30 minutes later the mice are suspended by their tails for six minutes. The amount of time that a mouse is immobile (i.e. is not displaying escape behavior) during the six minutes is measured to provide an immobility time. The administration of anti-depressant drugs such as imipramine reduces immobility time. Therefore, if a reduction of immobility time is observed when testing a test substance, it can be concluded that the test substance has anti-depressant properties. Immobility is considered a despair state, and, therefore, a reduction of immobility time also indicates an increase in motivation (i.e., that the test substance has motivation-increasing properties).

### 6.1.2. Forced Swimming Test

The forced swimming test, like the tail suspension test, is an experimental method used to screen potential antidepressant drugs (Can et al., 2012 J Vis Exp., 59:e3638). In the test as implemented in the Examples, a test substance is orally administered to mice (ddY mice, males, 24˜30 g) and 30 minutes later the mice are placed in an impossible to escape water bath for eight minutes. The amount of time that a mouse is immobile during the eight minutes is measured to provide an immobility time.

Like the tail suspension test, the administration of antidepressants, such as imipramine, reduces the immobility time in the forced swimming test. Therefore, if a reduction of immobility time is observed when testing a test substance, it can be it can be concluded that the test substance has anti-depressant properties. A reduction of immobility time also indicates an increase in motivation (i.e., that the test substance has motivation-increasing properties).

### 6.1.3. Elevated Plus Maze Test

The elevated plus maze test is a widely used test for measuring anxiety-like behavior in mice and is based on the natural aversion of mice for open and elevated areas (Komada et al., 2008 J. Vis Exp., 22:e1088). The test can be used to assess the anti-anxiety effects of test substances. In the test as implemented in the Examples, an apparatus consisting of two open arms (25 cm×5 cm) and two closed arms (25 cm×5 cm×15 cm) is positioned 50 cm above the floor. Despite the high position, a mouse can walk safely around the closed arms due to the high (15 cm) wall. On the other hand, a mouse walking the open arms feels a sense of anxiety because there is no wall protecting the mouse from falling. Thus, the longer the mouse is in the open arms, or the greater the frequency of entrance into the open arms, as compared to control, is an indication of anxiolytic activity of the test substance.

Mice (ddY mice, males, 24˜30 g) are administered a test substances 30 minutes prior to testing. At the beginning of the test, a mouse is placed on the apparatus facing one of the open arms. During the 5-minute test time, the cumulative time spent in the open arms (time in open arms), the number of times a visit to the open arms is made (visit to open arms), and the total number of times a visit to one of the arms is made (total visits) is recorded.

### 6.1.4. Open Field Test

The open field test is used to measure anxiety-like behavior in mice (Bailey and Crawley, 2009, Anxiety-Related Behaviors in Mice, Ch. 5 in Methods of Behavior Analysis in Neuroscience, 2^{nd} edition, CRC Press, Boca Raton Fla.). Mice prefer the peripheral part of an enclosed area (such as an enclosed circle), and searching behavior to the central part is typically very small. In the test as implemented in the Examples, mice are administered a test substance 30 minutes prior to testing.

At the start of the test, mice are placed in a circular chamber and their movement is observed for five minutes. The amount of time spent in the center circle of the chamber (12 cm), the number of visits to the center circle of the chamber, and the total distance traveled is measured. In mice administered the test substance, a higher proportion of time spent in the center circle as compared to control indicates anxiolytic activity of the test substance.

### 6.2. Example 1: Tail Suspension Test with Peptide LSSTQAQQSY (SEQ ID NO:1)

The peptide LSSTQAQQSY (SEQ ID NO:1) was tested in a tail suspension test using mice orally administered 0.1 mg/kg, 0.3 mg/kg, 1 mg/kg or 3 mg/kg of the peptide (n=8-10).

The results are shown in FIG. 1. Immobility time was significantly reduced in the 0.3 mg/kg, 1 mg/kg, and 3 mg/kg groups as compared to saline control.

### 6.3. Example 2: Forced Swimming Test with Peptide LSSTQAQQSY (SEQ ID NO:1)

The peptide LSSTQAQQSY (SEQ ID NO:1) was tested in a forced swimming test using mice orally administered 0.3 mg/kg or 1 mg/kg of the peptide (n=12-13).

The results are shown in FIG. 2. Immobility time was significantly reduced in 1 mg/kg group as compared to saline control.

### 6.4. Example 3: Tail Suspension Test with Peptides LSSTQAQQSY (SEQ ID NO:1), LSSTQAQQS (SEQ ID NO:2), SSTQAQQSY (SEQ ID NO:3), LSSTQ (SEQ ID NO:4), and AQQSY (SEQ ID NO:5)

Peptides LSSTQAQQSY (SEQ ID NO:1), LSSTQAQQS (SEQ ID NO:2) (corresponding to the first nine amino acids of SEQ ID NO:1), SSTQAQQSY (SEQ ID NO:3) (corresponding to the last nine amino acids of SEQ ID NO:1), LSSTQ (SEQ ID NO:4) (corresponding to the first five amino acids of SEQ ID NO:1), and AQQSY (SEQ ID NO:5) (corresponding to the last five amino acids of SEQ ID NO:1) were tested in a tail suspension test using mice orally administered 0.3 mg/kg of the peptides (n=5-6 or 18-19).

The results are shown in FIG. 3. Immobility time was reduced in all of the test groups as compared to control, indicating that each peptide has antidepressant and motivation-increasing properties. The results were only statistically significant for the full length peptide LSSTQAQQSY (SEQ ID NO:1). The inventors had the insight that a more significant effect can be seen at higher doses of the shorter peptides (e.g., at 1 mg/kg, 3 mg/kg, or 10 mg/kg).

### 6.5. Example 4: Elevated Plus Maze Test with Peptide LSSTQAQQSY (SEQ ID NO:1)

The peptide LSSTQAQQSY (SEQ ID NO:1) was tested in an elevated plus maze test using mice orally administered 0.1 mg/kg, 0.3 mg/kg, or 1 mg/kg of the peptide (n=18-19).

The results are shown in FIG. 4. The percentage of time spent in the open arms was increased in all groups, and rose to the level of statistical significance for the 1 mg/kg group.

### 6.6. Example 5: Open Field Test with Peptide LSSTQAQQSY (SEQ ID NO:1)

The peptide LSSTQAQQSY (SEQ ID NO:1) was tested in an open field test using mice orally administered 0.1 mg/kg, 0.3 mg/kg, or 1 mg/kg of the peptide (n=4-5).

The results are shown in FIG. 5. The amount of time that the mice spent in the center 12 cm circle was increased in all groups, and rose to the level of statistical significance in the 1 mg/kg group. A similar result was observed for the number of visits to the center 12 cm circle. On the other hand, the overall locomotor activity was not significantly different between the groups.

### 6.7. Example 6: Tail Suspension Test with the Peptides LSSTQAQQSY (SEQ ID NO:1), LSSTQAQQSW (SEQ ID NO:6), and LSSTQAQQSF (SEQ ID NO:7), and the C-Terminal Amidated Peptide Conjugate LSSTQAQQSY-NH₂ (SEQ ID NO:36)

Peptides LSSTQAQQSY (SEQ ID NO:1), LSSTQAQQSW (SEQ ID NO: 6) and LSSTQAQQSF (SEQ ID NO: 7), and C-terminal amidated peptide conjugate LSSTQAQQSY-NH₂ (SEQ ID NO:36) were tested in a tail suspension test using mice orally administered 0.3 mg/kg of the peptides (n=11-12).

The results are shown in FIG. 6. All of the peptides and the peptide conjugate significantly decreased immobility time, indicating that modifying the C-terminal end of the peptide LSSTQAQQSY (SEQ ID NO:1) by substituting the aromatic side chain (Y to W or F) or by adding an amino group maintains the anti-depressant and motivation-increasing activity of the peptide.

### 6.8. Example 7: Tail Suspension Test with Five, Six, Seven, Eight, and Nine Amino Acid Peptides

Five, six, seven, eight, and nine amino acid peptides SSTQA (SEQ ID NO:8), STQAQ (SEQ ID NO:9), TQAQQ (SEQ ID NO:10), QAQQS (SEQ ID NO: 11), AQQSW (SEQ ID NO:12), or AQQSF (SEQ ID NO:13), LSSTQA (SEQ ID NO:14), SSTQAQ (SEQ ID NO:15), STQAQQ (SEQ ID NO:16), TQAQQS (SEQ ID NO:17), QAQQSY (SEQ ID NO:18), QAQQSW (SEQ ID NO:19), and QAQQSF (SEQ ID NO:20), LSSTQAQ (SEQ ID NO:21), SSTQAQQ (SEQ ID NO:22), STQAQQS (SEQ ID NO:23), TQAQQSY (SEQ ID NO:24), TQAQQSW (SEQ ID NO:25), TQAQQSF (SEQ ID NO:26), LSSTQAQQ (SEQ ID NO:27), SSTQAQQS (SEQ ID NO:28), STQAQQSY (SEQ ID NO:29), STQAQQSW (SEQ ID NO:30), STQAQQSF (SEQ ID NO:31), SSTQAQQSW (SEQ ID NO:32) and SSTQAQQSF (SEQ ID NO:33) are tested in a tail suspension test using mice orally administered 1 mg/kg, 3 mg/kg, or 10 mg/kg of one of the peptides.

A decrease in immobility time is observed for the mice administered a five, six, seven, eight, or nine amino acid peptide. An increased response is observed with increasing peptide dose.

### 6.9. Example 8: Forced Swimming Test with Five, Six, Seven, Eight, and Nine Amino Acid Peptides

Five, six, seven, eight, and nine amino acid peptides SSTQA (SEQ ID NO:8), STQAQ (SEQ ID NO:9), TQAQQ (SEQ ID NO:10), QAQQS (SEQ ID NO: 11), AQQSW (SEQ ID NO:12), or AQQSF (SEQ ID NO:13), LSSTQA (SEQ ID NO:14), SSTQAQ (SEQ ID NO:15), STQAQQ (SEQ ID NO:16), TQAQQS (SEQ ID NO:17), QAQQSY (SEQ ID NO:18), QAQQSW (SEQ ID NO:19), and QAQQSF (SEQ ID NO:20), LSSTQAQ (SEQ ID NO:21), SSTQAQQ (SEQ ID NO:22), STQAQQS (SEQ ID NO:23), TQAQQSY (SEQ ID NO:24), TQAQQSW (SEQ ID NO:25), TQAQQSF (SEQ ID NO:26), LSSTQAQQ (SEQ ID NO:27), SSTQAQQS (SEQ ID NO:28), STQAQQSY (SEQ ID NO:29), STQAQQSW (SEQ ID NO:30), STQAQQSF (SEQ ID NO:31), SSTQAQQSW (SEQ ID NO:32) and SSTQAQQSF (SEQ ID NO:33) are tested in a forced swimming test using mice orally administered 1 mg/kg, 3 mg/kg, or 10 mg/kg of one of the peptides.

A decrease in immobility time is observed for the mice administered a five, six, seven, eight, or nine amino acid peptide. An increased response is observed with increasing peptide dose.

### 6.10. Example 9: Elevated Plus Maze Test with Five, Six, Seven, Eight, and Nine Amino Acid Peptides

Five, six, seven, eight, and nine amino acid peptides SSTQA (SEQ ID NO:8), STQAQ (SEQ ID NO:9), TQAQQ (SEQ ID NO:10), QAQQS (SEQ ID NO: 11), AQQSW (SEQ ID NO:12), or AQQSF (SEQ ID NO:13), LSSTQA (SEQ ID NO:14), SSTQAQ (SEQ ID NO:15), STQAQQ (SEQ ID NO:16), TQAQQS (SEQ ID NO:17), QAQQSY (SEQ ID NO:18), QAQQSW (SEQ ID NO:19), and QAQQSF (SEQ ID NO:20), LSSTQAQ (SEQ ID NO:21), SSTQAQQ (SEQ ID NO:22), STQAQQS (SEQ ID NO:23), TQAQQSY (SEQ ID NO:24), TQAQQSW (SEQ ID NO:25), TQAQQSF (SEQ ID NO:26), LSSTQAQQ (SEQ ID NO:27), SSTQAQQS (SEQ ID NO:28), STQAQQSY (SEQ ID NO:29), STQAQQSW (SEQ ID NO:30), STQAQQSF (SEQ ID NO:31), SSTQAQQSW (SEQ ID NO:32) and SSTQAQQSF (SEQ ID NO:33) are tested in an elevated plus maze test using mice orally administered 1 mg/kg, 3 mg/kg, or 10 mg/kg of one of the peptides.

The percentage of time spent in the open arms is increased for the mice administered a five, six, seven, eight, or nine amino acid peptide. An increased response is observed with increasing peptide dose.

### 6.11. Example 10: Open Field Test with Five, Six, Seven, Eight, and Nine Amino Acid Peptides

Five, six, seven, eight, and nine amino acid peptides SSTQA (SEQ ID NO:8), STQAQ (SEQ ID NO:9), TQAQQ (SEQ ID NO:10), QAQQS (SEQ ID NO: 11), AQQSW (SEQ ID NO:12), or AQQSF (SEQ ID NO:13), LSSTQA (SEQ ID NO:14), SSTQAQ (SEQ ID NO:15), STQAQQ (SEQ ID NO:16), TQAQQS (SEQ ID NO:17), QAQQSY (SEQ ID NO:18), QAQQSW (SEQ ID NO:19), and QAQQSF (SEQ ID NO:20), LSSTQAQ (SEQ ID NO:21), SSTQAQQ (SEQ ID NO:22), STQAQQS (SEQ ID NO:23), TQAQQSY (SEQ ID NO:24), TQAQQSW (SEQ ID NO:25), TQAQQSF (SEQ ID NO:26), LSSTQAQQ (SEQ ID NO:27), SSTQAQQS (SEQ ID NO:28), STQAQQSY (SEQ ID NO:29), STQAQQSW (SEQ ID NO:30), STQAQQSF (SEQ ID NO:31), SSTQAQQSW (SEQ ID NO:32) and SSTQAQQSF (SEQ ID NO:33) are tested in an open field test using mice orally administered 1 mg/kg, 3 mg/kg, or 10 mg/kg of one of the peptides.

The amount of time that the mice spend in the center 12 cm circle is increased for the mice administered a five, six, seven, eight, or nine amino acid peptide. An increased response is observed with increasing peptide dose.

### 6.12. Example 11: Examination of Mechanism of Action

The peptide LSSTQAQQSY (SEQ ID NO:1) was orally administered at 0.3 mg/kg to vagotomized mice and sham operated mice one week after surgery to investigate the mechanism of action of the peptide. The mice were then subjected to a tail suspension test.

The results are shown in FIG. 7. The vagotomy abolished anti-depressant-like effect of the orally administered peptide. These results suggest that the peptide acts on the gut, and its signal is transferred to the central nervous system via the vagus nerve. The inventors believe that the compounds of the disclosure are likely to share the same mechanism of action as the peptide LSSTQAQQSY (SEQ ID NO:1).

### 6.13. Example 12: Investigation of Mediators Involved in the Anti-Depressant Effects of the Peptide LSSTQAQQSY (SEQ ID NO:1)

The peptide LSSTQAQQSY (SEQ ID NO:1) was orally administered to mice at 0.3 mg/kg, alone and in combination with orally administered antagonists of serotonin 5-HT_{1A}, dopamine D₁, or GABA_{A} receptors and subjected to tail suspension test to investigate the mediators involved in the anti-depressant effects of the peptide. WAY100135, an antagonist of the 5-HT_{1A} receptor was dosed at 10 mg/kg. SCH23390, an antagonist of the dopamine D₁ receptor was dosed at 30 μg/kg. Bicuculline, an antagonist of the GABA_{A} receptor, was dosed at 30 mg/kg.

The results are shown in FIG. 8. The peptide-induced antidepressant-like effect was blocked by the antagonists of serotonin 5-HT_{1A}, dopamine D₁, and GABA_{A} receptors. The peptide exhibited no affinity for these receptors, suggesting that it stimulates the release of these neurotransmitters. It was also determined using selective agonists and antagonists (data not shown) that the order of the receptor activation is 5-HT_{1A}, D₁, and GABA_{A}. Taken together, the peptide appears to exhibit an antidepressant-like effect after oral administration via activation of the brain-gut axis, and of 5-HT_{1A}, D₁, and GABA_{A} systems. The inventors believe that the compounds of the disclosure are likely to share the same mechanism of action as the peptide LSSTQAQQSY (SEQ ID NO:1).

### 6.14. Example 13: Tail Suspension Test with Peptides LSSTQAQQ (SEQ ID NO:27), SSTQAQQS (SEQ ID NO:28), and STQAQQSY (SEQ ID NO:29)

Peptides LSSTQAQQ (SEQ ID NO:27), SSTQAQQS (SEQ ID NO:28), and STQAQQSY (SEQ ID NO:29) were tested in a tail suspension test using mice orally administered 0.3 mg/kg of the peptides (n=5-6).

The results are shown in FIG. 9. All of the peptides decreased immobility time compared to control.

### 6.15. Example 14: Tail Suspension Test with Peptides LSSTQAQQSYW (SEQ ID NO:37), WLSSTQAQQSY (SEQ ID NO:38), WLSSTQAQQSYW (SEQ ID NO:39), WLSSTQ (SEQ ID NO:40), and AQQSYW (SEQ ID NO:41)

Peptides LSSTQAQQSYW (SEQ ID NO:37), WLSSTQAQQSY (SEQ ID NO:38), WLSSTQAQQSYW (SEQ ID NO:39), WLSSTQ (SEQ ID NO:40), and AQQSYW (SEQ ID NO:41) were tested in a tail suspension test using mice orally administered 0.3 mg/kg of the peptides (n=3-5).

The results are shown in FIG. 10. All of the peptides decreased immobility time compared to control.

### 6.16. Example 15: Tail Suspension Test with Peptides ESFFLSSTQAQQSY (SEQ ID NO:42), LSSTQAQQSYLQGF (SEQ ID NO:43), and FFLSSTQAQQSYLQ (SEQ ID NO:44)

Peptides ESFFLSSTQAQQSY (SEQ ID NO:42), LSSTQAQQSYLQGF (SEQ ID NO:43), and FFLSSTQAQQSYLQ (SEQ ID NO:44) were tested in a tail suspension test using mice orally administered 0.3 mg/kg of the peptides (n=6-7).

The results are shown in FIG. 11. Peptide FFLSSTQAQQSYLQ (SEQ ID NO:44) decreased immobility time compared to control.

### 6.17. Example 16: Tail Suspension Test with Peptides SSTQAQQS (SEQ ID NO:28), LSSTQAQQSYW (SEQ ID NO:37) and WLSSTQ (SEQ ID NO:40)

Peptides SSTQAQQS (SEQ ID NO:28), LSSTQAQQSYW (SEQ ID NO:37) and WLSSTQ (SEQ ID NO:40) were retested in a tail suspension test using mice orally administered 0.3 mg/kg of the peptides (n=5-7).

The results are shown in FIG. 12. All of the peptides decreased immobility time compared to control.

### 6.18. Example 17: Analysis of Tail Suspension Test Data

The tail suspension test data from Examples 1, 3, 6, 13, 14, 15, and 16 were analyzed to identify correlations between sequence and activity. The tail suspension test data for the peptides were first normalized as a percentage of immobility time of the control in their respective study, such that lower percentages reflect greater activity. The results are shown in Table 1:

TABLE 1
Immobility times normalized to control

| | Immobility time for peptides dosed at 0.3 | | | | | | | |
|---|---|---|---|---|---|---|---|---|
| | mg/kg as percent of control immobility time | | | | | | | |
| | Example | | | | | | | |
| | | 3 | 3 | | | | | |
| SEQ | | (FIG. | (FIG. | | | | | |
| ID NO. | 1 | 3A) | 3B) | 6 | 13 | 14 | 15 | 16 |
| 1 | 44.13 | 42.41 | 64.04 | | | | | |
| 2 | | 87.31 | | | | | | |
| 3 | | 67.47 | | | | | | |
| 4 | | | 81.53 | | | | | |
| 5 | | | 86.87 | | | | | |
| 6 | | | | 38.41 | | | | |
| 7 | | | | 42.05 | | | | |
| 27 | | | | | 76.65 | | | |
| 28 | | | | | 60.34 | | | 57.29 |
| 29 | | | | | 74.44 | | | |
| 36 | | | | 55.27 | | | | |
| 37 | | | | | | 39.08 | | 58.54 |
| 38 | | | | | | 72.27 | | |
| 39 | | | | | | 90.94 | | |
| 40 | | | | | | 55.34 | | 88.09 |
| 41 | | | | | | 97.33 | | |
| 42 | | | | | | | 119.64 | |
| 43 | | | | | | | 109.70 | |
| 44 | | | | | | | 82.98 | |

The normalized data for each of the peptides was then compared to the normalized data for peptide LSSTQAQQSY (SEQ ID NO:1) to rank the relative activities of the peptides. The percent reduction of control immobility was used to assign an activity rating of excellent, good, moderate, or low to each peptide. For peptides that were tested more than once (including SEQ ID NO:1, the reference peptide), the average reduction of control immobility was used in these calculations. The resulting activity ratings, together with amino acid alignments in which amino acid changes from SEQ ID NO:1 are underlined, are shown in Table 2.

TABLE 2
Peptide activity ratings

| | | % of | | |
|---|---|---|---|---|
| SEQ | | SEQ ID | | |
| ID | | NO: 1 | | Activity |
| NO. | Sequence | activity | Rank | Rating |
| 6 | LSSTQAQQSW | 122.71 | 1 | Excellent |
| 7 | LSSTQAQQSF | 115.46 | 2 | Excellent |
| 37 | LSSTQAQQSYW | 97.25 | 4 | Excellent |
| 1 | LSSTQAQQSY | 100 | 3 | Excellent |
| 36 | LSSTQAQQSY* | 89.12 | 5 | Excellent |
| 28 | SSTQAQQS | 82.07 | 6 | Excellent |
| 3 | SSTQAQQSY | 64.81 | 7 | Excellent |
| 38 | WLSSTQAQQSY | 55.25 | 9 | Good |
| 29 | STQAQQSY | 50.93 | 10 | Good |
| 40 | WLSSTQ | 56.36 | 8 | Good |
| 27 | LSSTQAQQ | 46.52 | 11 | Good |
| 4 | LSSTQ | 36.80 | 12 | Good |
| 44 | FFLSSTQAQQSYLQ | 33.91 | 13 | Good |
| 5 | AQQSY | 26.16 | 14 | Moderate |
| 2 | LSSTQAQQS | 25.28 | 15 | Moderate |
| 39 | WLSSTQAQQSYW | 18.05 | 16 | Moderate |
| 41 | AQQSYW | 5.32 | 17 | Low |
| 43 | LSSTQAQQSYLQGF | -19.33 | 18 | Low |
| 42 | ESFFLSSTQAQQSY | -39.13 | 19 | Low |

Without being bound by theory, the inventors believe that peptides comprising a core amino acid sequence of SSTQAQQS (SEQ ID NO:28) and having a total length of 8 to 12 amino acids (and more particularly 8 to 10 amino acids) are generally more active than peptides not comprising the SSTQAQQS (SEQ ID NO:28) core sequence, that an aromatic amino acid (Y, W or F) at one or both termini, particularly the C-terminus, increases activity, and that activity generally gradually decreases as peptide length is altered to fall outside these ranges.

### 7. SPECIFIC EMBODIMENTS

The present disclosure is exemplified by the specific embodiments below.

1. A compound that is a peptide or a salt thereof, wherein the peptide:
a. is 5 to 15 amino acids in length;
b. has an amino acid sequence comprising at least 5 consecutive amino acids from the amino acid sequence LSSTQAQQSX₁ (SEQ ID NO:34); and
c. does not consist of the amino acid sequence LSSTQAQQSY (SEQ ID NO:1), LSSTQAQQS (SEQ ID NO:2), SSTQAQQSY (SEQ ID NO:3), LSSTQ (SEQ ID NO:4), AQQSY (SEQ ID NO:5), LSSTQAQQSW (SEQ ID NO:6), or LSSTQAQQSF (SEQ ID NO:7).
2. The compound of embodiment 1, wherein the peptide comprises or consists of the amino acid sequence SSTQA (SEQ ID NO:8).
3. The compound of embodiment 1, wherein the peptide comprises or consists of the amino acid sequence STQAQ (SEQ ID NO:9).
4. The compound of embodiment 1, wherein the peptide comprises or consists of the amino acid sequence TQAQQ (SEQ ID NO:10).
5. The compound of embodiment 1, wherein the peptide comprises or consists of the amino acid sequence QAQQS (SEQ ID NO: 11).
6. The compound of embodiment 1, wherein the peptide comprises or consists of the amino acid sequence AQQSW (SEQ ID NO:12).
7. The compound of embodiment 1, wherein the peptide comprises or consists of the amino acid sequence or AQQSF (SEQ ID NO:13).
8. The compound of embodiment 1, wherein the peptide comprises the amino acid sequence LSSTQ (SEQ ID NO:4).
9. The compound of embodiment 1, wherein the peptide comprises the amino acid sequence AQQSY (SEQ ID NO:5).
10. The compound of embodiment 1, wherein the peptide comprises or consists of the amino acid sequence LSSTQA (SEQ ID NO:14).
11. The compound of embodiment 1, wherein the peptide comprises or consists of the amino acid sequence SSTQAQ (SEQ ID NO:15).
12. The compound of embodiment 1, wherein the peptide comprises or consists of the amino acid sequence STQAQQ (SEQ ID NO:16).
13. The compound of embodiment 1, wherein the peptide comprises or consists of the amino acid sequence TQAQQS (SEQ ID NO:17).
14. The compound of embodiment 1, wherein the peptide comprises or consists of the amino acid sequence QAQQSY (SEQ ID NO:18).
15. The compound of embodiment 1, wherein the peptide comprises or consists of the amino acid sequence QAQQSW (SEQ ID NO:19).
16. The compound of embodiment 1, wherein the peptide comprises or consists of the amino acid sequence QAQQSF (SEQ ID NO:20).
17. The compound of embodiment 1, wherein the peptide comprises or consists of the amino acid sequence LSSTQAQ (SEQ ID NO:21).
18. The compound of embodiment 1, wherein the peptide comprises or consists of the amino acid sequence SSTQAQQ (SEQ ID NO:22).
19. The compound of embodiment 1, wherein the peptide comprises or consists of the amino acid sequence STQAQQS (SEQ ID NO:23).
20. The compound of embodiment 1, wherein the peptide comprises or consists of the amino acid sequence TQAQQSY (SEQ ID NO:24).
21. The compound of embodiment 1, wherein the peptide comprises or consists of the amino acid sequence TQAQQSW (SEQ ID NO:25).
22. The compound of embodiment 1, wherein the peptide comprises or consists of the amino acid sequence TQAQQSF (SEQ ID NO:26).
23. The compound of embodiment 1, wherein the peptide comprises or consists of the amino acid sequence LSSTQAQQ (SEQ ID NO:27).
24. The compound of embodiment 1, wherein the peptide comprises or consists of the amino acid sequence SSTQAQQS (SEQ ID NO:28).
25. The compound of embodiment 1, wherein the peptide comprises or consists of the amino acid sequence STQAQQSY (SEQ ID NO:29).
26. The compound of embodiment 1, wherein the peptide comprises or consists of the amino acid sequence STQAQQSW (SEQ ID NO:30).
27. The compound of embodiment 1, wherein the peptide comprises or consists of the amino acid sequence STQAQQSF (SEQ ID NO:31).
28. The compound of embodiment 1, wherein the peptide comprises or consists of the amino acid sequence SSTQAQQSW (SEQ ID NO:32).
29. The compound of embodiment 1, wherein the peptide comprises or consists of the amino acid sequence SSTQAQQSF (SEQ ID NO:33).
30. The compound of embodiment 1, wherein the peptide comprises the amino acid sequence LSSTQAQQS (SEQ ID NO:2).
31. The compound of embodiment 1, wherein the peptide comprises the amino acid sequence SSTQAQQSY (SEQ ID NO:3).
32. The compound of embodiment 1, wherein the peptide comprises the amino acid sequence LSSTQAQQSY (SEQ ID NO:1) 33. The compound of embodiment 1, wherein the peptide comprises the amino acid sequence LSSTQAQQSW (SEQ ID NO:6).
34. The compound of embodiment 1, wherein the peptide comprises the amino acid sequence LSSTQAQQSF (SEQ ID NO:7).
35. The compound of any one of embodiments 1 to 7, wherein the peptide is 5 amino acids is length.
36. The compound of any one of embodiments 1 to 16, wherein the peptide is 6 amino acids in length.
37. The compound of any one of embodiments 1 to 22, wherein the peptide is 7 amino acids in length.
38. The compound of any one of embodiments 1 to 27, wherein the peptide is 8 amino acids in length.
39. The compound of any one of embodiments 1 to 29, wherein the peptide is 9 amino acids in length.
40. The compound of any one of embodiments 1 to 31, wherein the peptide is 10 amino acids in length.
41. The compound of any one of embodiments 1 to 34, wherein the peptide is 11 amino acids in length.
42. The compound of any one of embodiments 1 to 34, wherein the peptide is 12 amino acids in length.
43. The compound of any one of embodiments 1 to 34, wherein the peptide is 13 amino acids in length.
44. The compound of any one of embodiments 1 to 34, wherein the peptide is 14 amino acids in length.
45. The compound of any one of embodiments 1 to 34, wherein the peptide is 15 amino acids in length.
46. A compound that is a conjugate or a salt thereof, the conjugate comprising:
a. a peptide moiety that:
i. is 5 to 15 amino acids in length; and
ii. comprises at least 5 consecutive amino acids from the amino acid sequence LSSTQAQQSX₁ (SEQ ID NO:34); and
iii. does not consist of the amino acid sequence LSSTQAQQSY (SEQ ID NO:1), LSSTQAQQSW (SEQ ID NO:6), or LSSTQAQQSF (SEQ ID NO:7);
attached to
b. one or more conjugate moieties.
47. The compound of embodiment 46, wherein the peptide moiety comprises or consists of the amino acid sequence LSSTQ (SEQ ID NO:4).
48. The compound of embodiment 46, wherein the peptide moiety comprises or consists of the amino acid sequence SSTQA (SEQ ID NO:8).
49. The compound of embodiment 46, wherein the peptide moiety comprises or consists of the amino acid sequence STQAQ (SEQ ID NO:9).
50. The compound of embodiment 46, wherein the peptide moiety comprises or consists of the amino acid sequence TQAQQ (SEQ ID NO:10).
51. The compound of embodiment 46, wherein the peptide moiety comprises or consists of the amino acid sequence QAQQS (SEQ ID NO: 11).
52. The compound of embodiment 46, wherein the peptide moiety comprises or consists of the amino acid sequence AQQSY (SEQ ID NO:5).
53. The compound of embodiment 46, wherein the peptide moiety comprises or consists of the amino acid sequence AQQSW (SEQ ID NO:12).
54. The compound of embodiment 46, wherein the peptide moiety comprises or consists of the amino acid sequence AQQSF (SEQ ID NO:13).
55. The compound of embodiment 46, wherein the peptide moiety comprises or consists of the amino acid sequence LSSTQA (SEQ ID NO:14).
56. The compound of embodiment 46, wherein the peptide moiety comprises or consists of the amino acid sequence SSTQAQ (SEQ ID NO:15).
57. The compound of embodiment 46, wherein the peptide moiety comprises or consists of the amino acid sequence STQAQQ (SEQ ID NO:16).
58. The compound of embodiment 46, wherein the peptide moiety comprises or consists of the amino acid sequence TQAQQS (SEQ ID NO:17).
59. The compound of embodiment 46, wherein the peptide moiety comprises or consists of the amino acid sequence QAQQSY (SEQ ID NO:18).
60. The compound of embodiment 46, wherein the peptide moiety comprises or consists of the amino acid sequence QAQQSW (SEQ ID NO:19).
61. The compound of embodiment 46, wherein the peptide moiety comprises or consists of the amino acid sequence QAQQSF (SEQ ID NO:20).
62. The compound of embodiment 46, wherein the peptide moiety comprises or consist of the amino acid sequence LSSTQAQ (SEQ ID NO:21).
63. The compound of embodiment 46, wherein the peptide moiety comprises or consists of the amino acid sequence SSTQAQQ (SEQ ID NO:22).
64. The compound of embodiment 46, wherein the peptide moiety comprises or consists of the amino acid sequence STQAQQS (SEQ ID NO:23).
65. The compound of embodiment 46, wherein the peptide moiety comprises or consists of the amino acid sequence TQAQQSY (SEQ ID NO:24).
66. The compound of embodiment 46, wherein the peptide moiety comprises or consists of the amino acid sequence TQAQQSW (SEQ ID NO:25).
67. The compound of embodiment 46, wherein the peptide moiety comprises or consists of the amino acid sequence TQAQQSF (SEQ ID NO:26).
68. The compound of embodiment 46, wherein the peptide moiety comprises or consists of the amino acid sequence LSSTQAQQ (SEQ ID NO:27).
69. The compound of embodiment 46, wherein the peptide moiety comprises or consists of the amino acid sequence SSTQAQQS (SEQ ID NO:28).
70. The compound of embodiment 46, wherein the peptide moiety comprises or consists of the amino acid sequence STQAQQSY (SEQ ID NO:29).
71. The compound of embodiment 46, wherein the peptide moiety comprises or consists of the amino acid sequence STQAQQSW (SEQ ID NO:30).
72. The compound of embodiment 46, wherein the peptide moiety comprises or consists of the amino acid sequence STQAQQSF (SEQ ID NO:31).
73. The compound of embodiment 46, wherein the peptide moiety comprises or consists of the amino acid sequence LSSTQAQQS (SEQ ID NO:2).
74. The compound of embodiment 46, wherein the peptide moiety comprises or consists of the amino acid sequence SSTQAQQSY (SEQ ID NO:3).
75. The compound of embodiment 46, wherein the peptide moiety comprises or consists of the amino acid sequence SSTQAQQSW (SEQ ID NO:32).
76. The compound of embodiment 46, wherein the peptide moiety comprises or consists of the amino acid sequence SSTQAQQSF (SEQ ID NO:33).
77. The compound of embodiment 46, wherein the peptide moiety comprises the amino acid sequence LSSTQAQQSY (SEQ ID NO:1).
78. The compound of embodiment 46, wherein the peptide moiety comprises the amino acid sequence LSSTQAQQSW (SEQ ID NO:6).
79. The compound of embodiment 46, wherein the peptide moiety comprises the amino acid sequence LSSTQAQQSF (SEQ ID NO:7).
80. The compound of any one of embodiments 46 to 54, wherein the peptide moiety is 5 amino acids is length.
81. The compound of any one of embodiments 46 to 61, wherein the peptide moiety is 6 amino acids in length.
82. The compound of any one of embodiments 46 to 67, wherein the peptide moiety is 7 amino acids in length.
83. The compound of any one of embodiments 46 to 72, wherein the peptide moiety is 8 amino acids in length.
84. The compound of any one of embodiments 46 to 76, wherein the peptide moiety is 9 amino acids in length.
85. The compound of any one of embodiments 46 to 76, wherein the peptide moiety is 10 amino acids in length.
86. The compound of any one of embodiments 46 to 79, wherein the peptide moiety is 11 amino acids in length.
87. The compound of any one of embodiments 46 to 79, wherein the peptide moiety is 12 amino acids in length.
88. The compound of any one of embodiments 46 to 79, wherein the peptide moiety is 13 amino acids in length.
89. The compound of any one of embodiments 46 to 79, wherein the peptide moiety is 14 amino acids in length.
90. The compound of any one of embodiments 46 to 79, wherein the peptide moiety is 15 amino acids in length.
91. The compound of any one of embodiments 46 to 90, wherein at least one of the one or more conjugate moieties comprises a polymer, an amino group, an acyl group, an alkyl group, a phosphate group, a lipid or a sugar.
92. The compound of embodiment 91, wherein at least one of the one or more conjugate moieties comprises a polymer.
93. The compound of embodiment 92, wherein the polymer comprises a polyethylene glycol, polyvinyl pyrrolidone, polylactic-co-glycolic acid, N-(2-hydroxypropyl) methacrylamide copolymer, polyglutamic acid, or a polysaccharide.
94. The compound of any one of embodiments 91 to 93, wherein at least one of the one or more conjugate moieties comprises an amine group.
95. The compound of embodiment 94, wherein the amine group is an amino group, an alkyl amino group, or a dialkyl amino group.
96. The compound of any one of embodiments 91 to 95, wherein at least one of the one or more conjugate moieties comprises an acyl group.
97. The compound of embodiment 96, wherein the acyl group is a formyl group or an acetyl group.
98. The compound of any one of embodiments 91 to 97, wherein at least one of the one or more conjugate moieties comprises an alkyl group.
99. The compound of embodiment 98, wherein the alkyl group is a methyl group or an ethyl group.
100. The compound of any one of embodiments 91 to 99, wherein at least one of the one or more conjugate moieties comprises a phosphate group.
101. The compound of any one of embodiments 91 to 100, wherein at least one of the one or more conjugate moieties comprises a lipid.
102. The compound of any one of embodiments 91 to 101, wherein at least one of the one or more conjugate moieties comprises a sugar.
103. The compound of any one of embodiments 46 to 93 or, to the extent dependent from embodiment 91, the compound of any one of embodiments 94 to 102, which comprises a single conjugate moiety.
104. The compound of embodiment 103, wherein the conjugate moiety is attached to the N-terminal amino acid of the peptide moiety.
105. The compound of embodiment 103, wherein the conjugate moiety is attached to the C-terminal amino acid of the peptide moiety.
106. The compound of any one of embodiments 46 to 102, which comprises more than one conjugate moiety.
107. The compound of embodiment 106, wherein all of the conjugate moieties are the same.
108. The compound of embodiment 106, wherein not all of the conjugate moieties are the same.
109. The compound of embodiment 106, wherein all of the conjugate moieties are different.
110. The compound of embodiment 106, which comprises a conjugate moiety attached to the N-terminal amino acid of the peptide moiety and a conjugate moiety attached to the C-terminal amino acid of the peptide moiety.
111. The compound of embodiment 110, wherein the conjugate moiety attached to the N-terminal amino acid of the peptide moiety is the same as the conjugate moiety attached to the C-terminal amino acid of the peptide moiety.
112. The compound of embodiment 110, wherein the conjugate moiety attached to the N-terminal amino acid of the peptide moiety is different from the conjugate moiety attached to the C-terminal amino acid of the peptide moiety.
113. The compound of any one of embodiments 1 to 112, which is a salt.
114. The compound of embodiment 113, wherein the salt is an acid addition salt.
115. The compound of embodiment 114, wherein the acid is:
a. hydrochloric acid;
b. sulfuric acid;
c. nitric acid;
d. phosphoric acid;
e. hydrobromic acid;
f. perchloric acid;
g. citric acid;
h. succinic acid;
i. maleic acid;
j. fumaric acid;
k. malic acid;
l. tartaric acid;
m. p-toluenesulfonic acid;
n. benzenesulfonic acid;
o. methanesulfonic acid; or
p. trifluoroacetic acid.
116. The compound of embodiment 113, wherein the salt is a base addition salt.
117. The compound of embodiment 116, wherein the base is:
a. sodium hydroxide;
b. potassium hydroxide;
c. lithium hydroxide;
d. calcium hydroxide; or
e. magnesium hydroxide.
118. A pharmaceutical composition comprising the compound of any one of embodiments 1 to 117 and one or more pharmaceutically acceptable carriers, diluents and/or excipients.
119. A food product comprising as an additive the compound of any one of embodiments 1 to 117.
120. The food product of embodiment 119, which is a dietary supplement.
121. The food product of embodiment 119, which is a functional food.
122. A method for treating a subject suffering from a mood disorder, an anxiety disorder, or a disorder of diminished motivation, comprising administering to the subject an effective amount of the compound of any one of embodiments 1 to 117 or the pharmaceutical composition of embodiment 118.
123. The method of embodiment 122, which comprises treating a subject suffering from a mood disorder.
124. The method of embodiment 122, wherein the mood disorder comprises depression, bipolar disorder, or adjustment disorder.
125. The method of embodiment 124, which comprises treating a subject suffering from depression.
126. The method of embodiment 124, which comprises treating a subject suffering from bipolar disorder.
127. The method of embodiment 124, which comprises treating a subject suffering from adjustment disorder.
128. The method of embodiment 122, which comprises treating a subject suffering from an anxiety disorder.
129. The method of embodiment 122, which comprises treating a subject suffering from a disorder of diminished motivation.
130. The method of embodiment 129, wherein the disorder of diminished motivation comprises apathy, abulia, or akinetic mutism.
131. The method of embodiment 130, which comprises treating a subject suffering from apathy.
132. The method of embodiment 130, which comprises treating a subject suffering from abulia.
133. The method of embodiment 130, which comprises treating a subject suffering from akinetic mutism.
134. The method of any one of embodiments 122 to 133, wherein the compound or pharmaceutical composition is administered orally.
135. The method of embodiment 134, wherein the amount of the compound administered to the subject per day is, or the amount of the pharmaceutical composition administered to the subject per day contains, 0.005 mg/kg to 500 mg/kg of the compound.
136. The method of embodiment 134, wherein the amount of the compound administered to the subject per day is, or the amount of the pharmaceutical composition administered to the subject per day contains, 0.005 mg/kg to 100 mg/kg of the compound.
137. The method of embodiment 134, wherein the amount of the compound administered to the subject per day is, or the amount of the pharmaceutical composition administered to the subject per day contains, 0.005 mg/kg to 30 mg/kg of the compound.
138. The method of embodiment 134, wherein the amount of the compound administered to the subject per day is, or the amount of the pharmaceutical composition administered to the subject per day contains, 0.005 mg/kg to 1 mg/kg of the compound.
139. The method of embodiment 134, wherein the amount of the compound administered to the subject per day is, or the amount of the pharmaceutical composition administered to the subject per day contains, 0.01 mg/kg to 30 mg/kg of the compound.
140. The method of embodiment 134, wherein the amount of the compound administered to the subject per day is, or the amount of the pharmaceutical composition administered to the subject per day contains, 0.01 mg/kg to 3 mg/kg of the compound.
141. The method of embodiment 134, wherein the amount of the compound administered to the subject per day is, or the amount of the pharmaceutical composition administered to the subject per day contains, 0.01 mg/kg to 1 mg/kg of the compound.
142. The method of embodiment 134, wherein the amount of the compound administered to the subject per day is, or the amount of the pharmaceutical composition administered to the subject per day contains, 0.02 mg/kg to 5 mg/kg of the compound.
143. The method of embodiment 134, wherein the amount of the compound administered to the subject per day is, or the amount of the pharmaceutical composition administered to the subject per day contains, 0.02 mg/kg to 2 mg/kg of the compound.
144. The method of embodiment 134, wherein the amount of the compound administered to the subject per day is, or the amount of the pharmaceutical composition administered to the subject per day contains, 0.02 mg/kg to 1 mg/kg of the compound.
145. The method of embodiment 134, wherein the amount of the compound administered to the subject per day is, or the amount of the pharmaceutical composition administered to the subject per day contains, 0.1 mg to 50 g.
146. The method of embodiment 134, wherein the amount of the compound administered to the subject per day is, or the amount of the pharmaceutical composition administered to the subject per day contains, 0.1 mg to 10 g of the compound.
147. The method of embodiment 134, wherein the amount of the compound administered to the subject per day is, or the amount of the pharmaceutical composition administered to the subject per day contains, 0.1 mg to 3 g of the compound.
148. The method of embodiment 134, wherein the amount of the compound administered to the subject per day is, or the amount of the pharmaceutical composition administered to the subject per day contains, 0.1 mg to 100 mg of the compound.
149. The method of embodiment 134, wherein the amount of the compound administered to the subject per day is, or the amount of the pharmaceutical composition administered to the subject per day contains, 0.1 mg to 1 mg of the compound.
150. The method of embodiment 134, wherein the amount of the compound administered to the subject per day is, or the amount of the pharmaceutical composition administered to the subject per day contains, 0.3 mg to 3 g of the compound.
151. The method of embodiment 134, wherein the amount of the compound administered to the subject per day is, or the amount of the pharmaceutical composition administered to the subject per day contains, 0.3 mg to 100 mg of the compound.
152. A method for treating or preventing a mood disorder, an anxiety disorder, or a disorder of diminished motivation, comprising administering to a subject prone to or suffering from a mood disorder, an anxiety disorder, or a disorder of diminished motivation an effective amount of one or more food products of any one of embodiments 119 to 121.
153. The method of embodiment 152, which comprises treating or preventing a mood disorder.
154. The method of embodiment 152, wherein the mood disorder comprises depression, bipolar disorder, or adjustment disorder.
155. The method of embodiment 154, which comprises treating or preventing depression.
156. The method of embodiment 154, which comprises treating or preventing bipolar disorder.
157. The method of embodiment 154, which comprises treating or preventing adjustment disorder.
158. The method of embodiment 152, which comprises treating or preventing an anxiety disorder.
159. The method of embodiment 152, which comprises treating or preventing a disorder of diminished motivation.
160. The method of embodiment 159, wherein the disorder of diminished motivation comprises apathy, abulia, or akinetic mutism.
161. The method of embodiment 160, which comprises treating or preventing apathy.
162. The method of embodiment 160, which comprises treating or preventing abulia.
163. The method of embodiment 160, which comprises treating or preventing akinetic mutism.
164. The method of any one of embodiments 152 to 163, wherein the amount of the one or more food products administered to the subject per day contains 0.005 mg/kg to 500 mg/kg of the compound.
165. The method of any one of embodiments 152 to 163, wherein the amount of the one or more food products administered to the subject per day contains 0.005 mg/kg to 100 mg/kg of the compound.
166. The method of any one of embodiments 152 to 163, wherein the amount of the one or more food products administered to the subject per day contains 0.005 mg/kg to 30 mg/kg of the compound.
167. The method of any one of embodiments 152 to 163, wherein the amount of the one or more food products administered to the subject per day contains 0.005 mg/kg to 1 mg/kg of the compound.
168. The method of any one of embodiments 152 to 163, wherein the amount of the one or more food products administered to the subject per day contains 0.01 mg/kg to 30 mg/kg of the compound.
169. The method of any one of embodiments 152 to 163, wherein the amount of the one or more food products administered to the subject per day contains 0.01 mg/kg to 3 mg/kg of the compound.
170. The method of any one of embodiments 152 to 163, wherein the amount of the one or more food products administered to the subject per day contains 0.01 mg/kg to 1 mg/kg of the compound.
171. The method of any one of embodiments 152 to 163, wherein the amount of the one or more food products administered to the subject per day contains 0.02 mg/kg to 5 mg/kg of the compound.
172. The method of any one of embodiments 152 to 163, wherein the amount of the one or more food products administered to the subject per day contains 0.02 mg/kg to 2 mg/kg of the compound.
173. The method of any one of embodiments 152 to 163, wherein the amount of the one or more food products administered to the subject per day contains 0.02 mg/kg to 1 mg/kg of the compound.
174. The method of any one of embodiments 152 to 163, wherein the amount of the one or more food products administered to the subject per day contains 0.1 mg to 50 g of the compound.
175. The method of any one of embodiments 152 to 163, wherein the amount of the one or more food products administered to the subject per day contains 0.1 mg to 10 g of the compound.
176. The method of any one of embodiments 152 to 163, wherein the amount of the one or more food products administered to the subject per day contains 0.1 mg to 3 g of the compound.
177. The method of any one of embodiments 152 to 163, wherein the amount of the one or more food products administered to the subject per day contains 0.1 mg to 100 mg of the compound.
178. The method of any one of embodiments 152 to 163, wherein the amount of the one or more food products administered to the subject per day contains 0.1 mg to 1 mg of the compound.
179. The method of any one of embodiments 152 to 163, wherein the amount of the one or more food products administered to the subject per day contains 0.3 mg to 3 g of the compound.
180. The method of any one of embodiments 152 to 163, wherein the amount of the one or more food products administered to the subject per day contains 0.3 mg to 100 mg of the compound.
181. A compound according to any one of embodiments 1 to 117 for use as a medicament.
182. A compound according to any one of embodiments 1 to 117 for use in a method for the treatment of a mood disorder.
183. The compound for use according to embodiment 182, wherein the mood disorder comprises depression, bipolar disorder, or adjustment disorder.
184. The compound for use according to embodiment 183, wherein the mood disorder comprises depression.
185. The compound for use according to embodiment 183, wherein the mood disorder comprises bipolar disorder.
186. The compound for use according to embodiment 183, wherein the mood disorder comprises adjustment disorder.
187. A compound according to any one of embodiments 1 to 117 for use in a method for the treatment of an anxiety disorder.
188. A compound according to any one of embodiments 1 to 117 for use in a method for the treatment of a disorder of diminished motivation.
189. The compound for use according to embodiment 188, wherein the disorder of diminished motivation comprises apathy, abulia, or akinetic mutism.
190. The compound for use according to embodiment 189, wherein the disorder of diminished motivation comprises apathy.
191. The compound for use according to embodiment 189, wherein the disorder of diminished motivation comprises abulia.
192. The compound for use according to embodiment 189, wherein the disorder of diminished motivation comprises akinetic mutism.
193. The compound for use according to any one of embodiments 182 to 192, wherein the method comprises orally administering an effective amount of the compound to a subject.
194. The compound for use according to embodiment 193, wherein the method comprises administering 0.005 mg/kg to 500 mg/kg of the compound to the subject per day.
195. The compound for use according to embodiment 193, wherein the method comprises administering 0.005 mg/kg to 100 mg/kg of the compound to the subject per day.
196. The compound for use according to embodiment 193, wherein the method comprises administering 0.005 mg/kg to 30 mg/kg of the compound to the subject per day.
197. The compound for use according to embodiment 193, wherein the method comprises administering 0.005 mg/kg to 1 mg/kg of the compound to the subject per day.
198. The compound for use according to embodiment 193, wherein the method comprises administering 0.01 mg/kg to 30 mg/kg of the compound to the subject per day.
199. The compound for use according to embodiment 193, wherein the method comprises administering 0.01 mg/kg to 3 mg/kg of the compound to the subject per day.
200. The compound for use according to embodiment 193, wherein the method comprises administering 0.01 mg/kg to 1 mg/kg of the compound to the subject per day.
201. The compound for use according to embodiment 193, wherein the method comprises administering 0.02 mg/kg to 5 mg/kg of the compound to the subject per day.
202. The compound for use according to embodiment 193, wherein the method comprises administering 0.02 mg/kg to 2 mg/kg of the compound to the subject per day.
203. The compound for use according to embodiment 193, wherein the method comprises administering 0.02 mg/kg to 1 mg/kg of the compound to the subject per day.
204. The compound for use according to embodiment 193, wherein the method comprises administering 0.1 mg to 50 g of the compound to the subject per day.
205. The compound for use according to embodiment 193, wherein the method comprises administering 0.1 mg to 10 g of the compound to the subject per day.
206. The compound for use according to embodiment 193, wherein the method comprises administering 0.1 mg to 3 g of the compound to the subject per day.
207. The compound for use according to embodiment 193, wherein the method comprises administering 0.1 mg to 100 mg of the compound to the subject per day.
208. The compound for use according to embodiment 193, wherein the method comprises administering 0.1 mg to 1 mg of the compound to the subject per day.
209. The compound for use according to embodiment 193, wherein the method comprises administering 0.3 mg to 3 g of the compound to the subject per day.
210. The compound for use according to embodiment 193, wherein the method comprises administering 0.3 mg to 100 mg of the compound to the subject per day.
211. A pharmaceutical composition according to embodiment 118 for use in a method for the treatment of a mood disorder.
212. The pharmaceutical composition for use according to embodiment 211, wherein the mood disorder comprises depression, bipolar disorder, or adjustment disorder.
213. The pharmaceutical composition for use according to embodiment 211, wherein the mood disorder comprises depression.
214. The pharmaceutical composition for use according to embodiment 211, wherein the mood disorder comprises bipolar disorder.
215. The pharmaceutical composition for use according to embodiment 211, wherein the mood disorder comprises adjustment disorder.
216. A pharmaceutical composition according to embodiment 118 for use in a method for the treatment of an anxiety disorder.
217. A pharmaceutical composition according to embodiment 118 for use in a method for the treatment of a disorder of diminished motivation.
218. The pharmaceutical composition for use according to embodiment 217, wherein the disorder of diminished motivation comprises apathy, abulia, or akinetic mutism.
219. The pharmaceutical composition for use according to embodiment 217, wherein the disorder of diminished motivation comprises apathy.
220. The pharmaceutical composition for use according to embodiment 217, wherein the disorder of diminished motivation comprises abulia.
221. The pharmaceutical composition for use according to embodiment 217, wherein the disorder of diminished motivation comprises akinetic mutism.
222. The pharmaceutical composition for use according to any one of embodiments 211 to 221, wherein the method comprises orally administering an effect amount of the pharmaceutical composition to a subject.
223. The pharmaceutical composition for use according to embodiment 222, wherein the method comprises administering an amount of the pharmaceutical composition to the subject per day that contains 0.005 mg/kg to 500 mg/kg of the compound.
224. The pharmaceutical composition for use according to embodiment 222, wherein the method comprises administering an amount of the pharmaceutical composition to the subject per day that contains 0.005 mg/kg to 100 mg/kg of the compound.
225. The pharmaceutical composition for use according to embodiment 222, wherein the method comprises administering an amount of the pharmaceutical composition to the subject per day that contains 0.005 mg/kg to 30 mg/kg of the compound.
226. The pharmaceutical composition for use according to embodiment 222, wherein the method comprises administering an amount of the pharmaceutical composition to the subject per day that contains 0.005 mg/kg to 1 mg/kg of the compound.
227. The pharmaceutical composition for use according to embodiment 222, wherein the method comprises administering an amount of the pharmaceutical composition to the subject per day that contains 0.01 mg/kg to 30 mg/kg of the compound.
228. The pharmaceutical composition for use according to embodiment 222, wherein the method comprises administering an amount of the pharmaceutical composition to the subject per day that contains 0.01 mg/kg to 3 mg/kg of the compound.
229. The pharmaceutical composition for use according to embodiment 222, wherein the method comprises administering an amount of the pharmaceutical composition to the subject per day that contains 0.01 mg/kg to 1 mg/kg of the compound.
230. The pharmaceutical composition for use according to embodiment 222, wherein the method comprises administering an amount of the pharmaceutical composition to the subject per day that contains 0.02 mg/kg to 5 mg/kg of the compound.
231. The pharmaceutical composition for use according to embodiment 222, wherein the method comprises administering an amount of the pharmaceutical composition to the subject per day that contains 0.02 mg/kg to 2 mg/kg of the compound.
232. The pharmaceutical composition for use according to embodiment 222, wherein the method comprises administering an amount of the pharmaceutical composition to the subject per day that contains 0.02 mg/kg to 1 mg/kg of the compound.
233. The pharmaceutical composition for use according to embodiment 222, wherein the method comprises administering an amount of the pharmaceutical composition to the subject per day that contains 0.1 mg to 50 g of the compound.
234. The pharmaceutical composition for use according to embodiment 222, wherein the method comprises administering an amount of the pharmaceutical composition to the subject per day that contains 0.1 mg to 10 g of the compound.
235. The pharmaceutical composition for use according to embodiment 222, wherein the method comprises administering an amount of the pharmaceutical composition to the subject per day that contains 0.1 mg to 3 g of the compound.
236. The pharmaceutical composition for use according to embodiment 222, wherein the method comprises administering an amount of the pharmaceutical composition to the subject per day that contains 0.1 mg to 100 mg of the compound.
237. The pharmaceutical composition for use according to embodiment 222, wherein the method comprises administering an amount of the pharmaceutical composition to the subject per day that contains 0.1 mg to 1 mg of the compound.
238. The pharmaceutical composition for use according to embodiment 222, wherein the method comprises administering an amount of the pharmaceutical composition to the subject per day that contains 0.3 mg to 3 g of the compound.
239. The pharmaceutical composition for use according to embodiment 222, wherein the method comprises administering an amount of the pharmaceutical composition to the subject per day that contains 0.3 mg to 100 mg of the compound.
240. A food product according to any one of embodiments 119 to 121 for use in a method for the treatment of or prevention of a mood disorder.
241. The food product for use according to embodiment 240, wherein the mood disorder comprises depression, bipolar disorder, or adjustment disorder.
242. The food product for use according to embodiment 241, wherein the mood disorder comprises depression.
243. The food product for use according to embodiment 241, wherein the mood disorder comprises bipolar disorder.
244. The food product for use according to embodiment 241, wherein the mood disorder comprises adjustment disorder.
245. A food product according to any one of embodiments 119 to 121 for use in a method for the treatment of or prevention of an anxiety disorder.
246. A food product for use according to any one of embodiments 119 to 121 for use in a method for the treatment of or prevention of a disorder of diminished motivation.
247. The food product for use according to embodiment 246, wherein the disorder of diminished motivation comprises apathy, abulia, or akinetic mutism.
248. The food product for use according to embodiment 247, wherein the disorder of diminished motivation comprises apathy.
249. The food product for use according to embodiment 247, wherein the disorder of diminished motivation comprises abulia.
250. The food product for use according to embodiment 247, wherein the disorder of diminished motivation comprises akinetic mutism.
251. The food product for use according to any one of embodiments 240 to 250, wherein the method comprises administering an amount of one or more food products according to any one of embodiments 119 to 121 to a subject, and wherein the amount of the one or more food products administered to the subject per day contains 0.005 mg/kg to 500 mg/kg of the compound.
252. The food product for use according to any one of embodiments 240 to 250, wherein the method comprises administering an amount of one or more food products according to any one of embodiments 119 to 121 to a subject, and wherein the amount of the one or more food products administered to the subject per day contains 0.005 mg/kg to 100 mg/kg of the compound.
253. The food product for use according to any one of embodiments 240 to 250, wherein the method comprises administering an amount of one or more food products according to any one of embodiments 119 to 121 to a subject, and wherein the amount of the one or more food products administered to the subject per day contains 0.005 mg/kg to 30 mg/kg of the compound.
254. The food product for use according to any one of embodiments 240 to 250, wherein the method comprises administering an amount of one or more food products according to any one of embodiments 119 to 121 to a subject, and wherein the amount of the one or more food products administered to the subject per day contains 0.005 mg/kg to 1 mg/kg of the compound.
255. The food product for use according to any one of embodiments 240 to 250, wherein the method comprises administering an amount of one or more food products according to any one of embodiments 119 to 121 to a subject, and wherein the amount of the one or more food products administered to the subject per day contains 0.01 mg/kg to 30 mg/kg of the compound.
256. The food product for use according to any one of embodiments 240 to 250, wherein the method comprises administering an amount of one or more food products according to any one of embodiments 119 to 121 to a subject, and wherein the amount of the one or more food products administered to the subject per day contains 0.01 mg/kg to 3 mg/kg of the compound.
257. The food product for use according to any one of embodiments 240 to 250, wherein the method comprises administering an amount of one or more food products according to any one of embodiments 119 to 121 to a subject, and wherein the amount of the one or more food products administered to the subject per day contains 0.01 mg/kg to 1 mg/kg of the compound.
258. The food product for use according to any one of embodiments 240 to 250, wherein the method comprises administering an amount of one or more food products according to any one of embodiments 119 to 121 to a subject, and wherein the amount of the one or more food products administered to the subject per day contains 0.02 mg/kg to 5 mg/kg of the compound.
259. The food product for use according to any one of embodiments 240 to 250, wherein the method comprises administering an amount of one or more food products according to any one of embodiments 119 to 121 to a subject, and wherein the amount of the one or more food products administered to the subject per day contains 0.02 mg/kg to 2 mg/kg of the compound.
260. The food product for use according to any one of embodiments 240 to 250, wherein the method comprises administering an amount of one or more food products according to any one of embodiments 119 to 121 to a subject, and wherein the amount of the one or more food products administered to the subject per day contains 0.02 mg/kg to 1 mg/kg of the compound.
261. The food product for use according to any one of embodiments 240 to 250, wherein the method comprises administering an amount of one or more food products according to any one of embodiments 119 to 121 to a subject, and wherein the amount of the one or more food products administered to the subject per day contains 0.1 mg to 50 g of the compound.
262. The food product for use according to any one of embodiments 240 to 250, wherein the method comprises administering an amount of one or more food products according to any one of embodiments 119 to 121 to a subject, and wherein the amount of the one or more food products administered to the subject per day contains 0.1 mg to 10 g of the compound.
263. The food product for use according to any one of embodiments 240 to 250, wherein the method comprises administering an amount of one or more food products according to any one of embodiments 119 to 121 to a subject, and wherein the amount of the one or more food products administered to the subject per day contains 0.1 mg to 3 g of the compound.
264. The food product for use according to any one of embodiments 240 to 250, wherein the method comprises administering an amount of one or more food products according to any one of embodiments 119 to 121 to a subject, and wherein the amount of the one or more food products administered to the subject per day contains 0.1 mg to 100 mg of the compound.
265. The food product for use according to any one of embodiments 240 to 250, wherein the method comprises administering an amount of one or more food products according to any one of embodiments 119 to 121 to a subject, and wherein the amount of the one or more food products administered to the subject per day contains 0.1 mg to 1 mg of the compound.
266. The food product for use according to any one of embodiments 240 to 250, wherein the method comprises administering an amount of one or more food products according to any one of embodiments 119 to 121 to a subject, and wherein the amount of the one or more food products administered to the subject per day contains 0.3 mg to 3 g of the compound.
267. The food product for use according to any one of embodiments 240 to 250, wherein the method comprises administering an amount of one or more food products according to any one of embodiments 119 to 121 to a subject, and wherein the amount of the one or more food products administered to the subject per day contains 0.3 mg to 100 mg of the compound.

While various specific embodiments have been illustrated and described, it will be appreciated that various changes can be made without departing from the spirit and scope of the disclosure(s).

### 8. SEQUENCE LISTING

TABLE 3

| Sequence | length | SEQ ID NO |
|---|---|---|
| LSSTQAQQSY | 10 | 1 |
| LSSTQAQQS | 9 | 2 |
| SSTQAQQSY | 9 | 3 |
| LSSTQ | 5 | 4 |
| AQQSY | 5 | 5 |
| LSSTQAQQSW | 10 | 6 |
| LSSTQAQQSF | 10 | 7 |
| SSTQA | 5 | 8 |
| STQAQ | 5 | 9 |
| TQAQQ | 5 | 10 |
| QAQQS | 5 | 11 |
| AQQSW | 5 | 12 |
| AQQSF | 5 | 13 |
| LSSTQA | 6 | 14 |
| SSTQAQ | 6 | 15 |
| STQAQQ | 6 | 16 |
| TQAQQS | 6 | 17 |
| QAQQSY | 6 | 18 |
| QAQQSW | 6 | 19 |
| QAQQSF | 6 | 20 |
| LSSTQAQ | 7 | 21 |
| SSTQAQQ | 7 | 22 |
| STQAQQS | 7 | 23 |
| TQAQQSY | 7 | 24 |

TABLE 4

| Sequence | length | SEQ ID NO |
|---|---|---|
| TQAQQSW | 7 | 25 |
| TQAQQSF | 7 | 26 |
| LSSTQAQQ | 8 | 27 |
| SSTQAQQS | 8 | 28 |
| STQAQQSY | 8 | 29 |
| STQAQQSW | 8 | 30 |
| STQAQQSF | 8 | 31 |
| SSTQAQQSW | 9 | 32 |
| SSTQAQQSF | 9 | 33 |
| LSSTQAQQSX₁, where X₁ is Y, | 10 | 34 |
| W, or F | | |
| NKPGRFESFFLSSTQAQQSX₁ | 30 | 35 |
| LQGFSKNILE, where X₁ is Y, | | |
| W, or F | | |
| LSSTQAQQSY-NH₂ | 10 | 36 |
| LSSTQAQQSYW | 11 | 37 |
| WLSSTQAQQSY | 11 | 38 |
| WLSSTQAQQSYW | 12 | 39 |
| WLSSTQ | 6 | 40 |
| AQQSYW | 6 | 41 |
| ESFFLSSTQAQQSY | 14 | 42 |
| LSSTQAQQSYLQGF | 14 | 43 |
| FFLSSTQAQQSYLQ | 14 | 44 |

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1-33. (canceled)

34. A method for treating a subject suffering from a mood disorder, an anxiety disorder, or a disorder of diminished motivation, comprising administering to the subject an effective amount of a compound that is a peptide or a salt thereof, wherein the amino acid sequence of the peptide:
a. consists of:
i. 5 to 10 consecutive amino acids from the amino acid sequence LSSTQAQQSX₁ (SEQ ID NO:34), where X₁ is Y, W, or F; and
ii. optionally, an additional aromatic amino acid at the N-terminus and/or an additional aromatic amino acid at the C-terminus of said 5 to 10 consecutive amino acids; and
b. does not consist of the amino acid sequence LSSTQAQQSY (SEQ ID NO:1), LSSTQAQQS (SEQ ID NO:2), SSTQAQQSY (SEQ ID NO:3), LSSTQ (SEQ ID NO:4), or AQQSY (SEQ ID NO:5), LSSTQAQQSW (SEQ ID NO:6), or LSSTQAQQSF (SEQ ID NO:7).

35. The method of claim 34, wherein the peptide is 5 amino acids is length.

36. The method of claim 34, wherein the peptide is 6 amino acids is length.

37. The method of claim 34, wherein the peptide is 7 amino acids is length.

38. The method of claim 34, wherein the peptide is 8 amino acids is length.

39. The method of claim 34, wherein the peptide is 9 amino acids in length.

40. The method of claim 34, wherein the peptide is 10 amino acids in length.

41. The method of claim 34, wherein the peptide is 11 amino acids in length.

42. The method of claim 34, wherein the peptide is 12 amino acids in length.

43. The method of claim 34, wherein the peptide comprises the amino acid sequence SSTQA (SEQ ID NO:8), STQAQ (SEQ ID NO:9), TQAQQ (SEQ ID NO:10), QAQQS (SEQ ID NO:11), AQQSW (SEQ ID NO:12), or AQQSF (SEQ ID NO:13).

44. The method of claim 34, wherein the peptide comprises the amino acid sequence LSSTQA (SEQ ID NO:14), SSTQAQ (SEQ ID NO:15), STQAQQ (SEQ ID NO:16), TQAQQS (SEQ ID NO:17), QAQQSY (SEQ ID NO:18), QAQQSW (SEQ ID NO:19), or QAQQSF (SEQ ID NO:20).

45. The method of claim 34, wherein the peptide comprises the amino acid sequence LSSTQAQ (SEQ ID NO:21), SSTQAQQ (SEQ ID NO:22), STQAQQS (SEQ ID NO:23), TQAQQSY (SEQ ID NO:24), TQAQQSW (SEQ ID NO:25), or TQAQQSF (SEQ ID NO:26).

46. The method of claim 34, wherein the peptide comprises the amino acid sequence LSSTQAQQ (SEQ ID NO:27), SSTQAQQS (SEQ ID NO:28), STQAQQSY (SEQ ID NO:29), STQAQQSW (SEQ ID NO:30), or STQAQQSF (SEQ ID NO:31).

47. The method of claim 34, wherein the peptide comprises the amino acid sequence SSTQAQQSW (SEQ ID NO:32) or SSTQAQQSF (SEQ ID NO:33).

48. The method of claim 34, wherein the subject is suffering from a mood disorder.

49. The method of claim 48, wherein the mood disorder is depression, bipolar disorder, or adjustment disorder.

50. The method of claim 34, wherein the subject is suffering from an anxiety disorder.

51. The method of claim 34, wherein the subject is suffering from a disorder of diminished motivation.

52. The method of claim 51, wherein the disorder of diminished motivation is apathy, abulia, or akinetic mutism.

53. The method of claim 34, wherein the compound is administered orally.


SEQUENCE LISTING (44 sequences)

SEQ ID NO: 1 - protein, 10 aa, Artificial Sequence
  Designed peptide
LSSTQAQQSY

SEQ ID NO: 2 - protein, 9 aa, Artificial Sequence
  Designed peptide
LSSTQAQQS

SEQ ID NO: 3 - protein, 9 aa, Artificial Sequence
  Designed peptide
SSTQAQQSY

SEQ ID NO: 4 - protein, 5 aa, Artificial Sequence
  Designed peptide
LSSTQ

SEQ ID NO: 5 - protein, 5 aa, Artificial Sequence
  Designed peptide
AQQSY

SEQ ID NO: 6 - protein, 10 aa, Artificial Sequence
  Designed peptide
LSSTQAQQSW

SEQ ID NO: 7 - protein, 10 aa, Artificial Sequence
  Designed peptide
LSSTQAQQSF

SEQ ID NO: 8 - protein, 5 aa, Artificial Sequence
  Designed peptide
SSTQA

SEQ ID NO: 9 - protein, 5 aa, Artificial Sequence
  Designed peptide
STQAQ

SEQ ID NO: 10 - protein, 5 aa, Artificial Sequence
  Designed peptide
TQAQQ

SEQ ID NO: 11 - protein, 5 aa, Artificial Sequence
  Designed peptide
QAQQS

SEQ ID NO: 12 - protein, 5 aa, Artificial Sequence
  Designed peptide
AQQSW

SEQ ID NO: 13 - protein, 5 aa, Artificial Sequence
  Designed peptide
AQQSF

SEQ ID NO: 14 - protein, 6 aa, Artificial Sequence
  Designed peptide
LSSTQA

SEQ ID NO: 15 - protein, 6 aa, Artificial Sequence
  Designed peptide
SSTQAQ

SEQ ID NO: 16 - protein, 6 aa, Artificial Sequence
  Designed peptide
STQAQQ

SEQ ID NO: 17 - protein, 6 aa, Artificial Sequence
  Designed peptide
TQAQQS

SEQ ID NO: 18 - protein, 6 aa, Artificial Sequence
  Designed peptide
QAQQSY

SEQ ID NO: 19 - protein, 6 aa, Artificial Sequence
  Designed peptide
QAQQSW

SEQ ID NO: 20 - protein, 6 aa, Artificial Sequence
  Designed peptide
QAQQSF

SEQ ID NO: 21 - protein, 7 aa, Artificial Sequence
  Designed peptide
LSSTQAQ

SEQ ID NO: 22 - protein, 7 aa, Artificial Sequence
  Designed peptide
SSTQAQQ

SEQ ID NO: 23 - protein, 7 aa, Artificial Sequence
  Designed peptide
STQAQQS

SEQ ID NO: 24 - protein, 7 aa, Artificial Sequence
  Designed peptide
TQAQQSY

SEQ ID NO: 25 - protein, 7 aa, Artificial Sequence
  Designed peptide
TQAQQSW

SEQ ID NO: 26 - protein, 7 aa, Artificial Sequence
  Designed peptide
TQAQQSF

SEQ ID NO: 27 - protein, 8 aa, Artificial Sequence
  Designed peptide
LSSTQAQQ

SEQ ID NO: 28 - protein, 8 aa, Artificial Sequence
  Designed peptide
SSTQAQQS

SEQ ID NO: 29 - protein, 8 aa, Artificial Sequence
  Designed peptide
STQAQQSY

SEQ ID NO: 30 - protein, 8 aa, Artificial Sequence
  Designed peptide
STQAQQSW

SEQ ID NO: 31 - protein, 8 aa, Artificial Sequence
  Designed peptide
STQAQQSF

SEQ ID NO: 32 - protein, 9 aa, Artificial Sequence
  Designed peptide
SSTQAQQSW

SEQ ID NO: 33 - protein, 9 aa, Artificial Sequence
  Designed peptide
SSTQAQQSF

SEQ ID NO: 34 - protein, 10 aa, Artificial Sequence
  Designed peptide
  MISC_FEATURE (10)..(10): Xaa(10)=Y, W, or F
LSSTQAQQSX

SEQ ID NO: 35 - protein, 30 aa, Artificial Sequence
  Designed peptide
  MISC_FEATURE (20)..(20): Xaa(20)=Y, W, or F
NKPGRFESFFLSSTQAQQSXLQGFSKNILE

SEQ ID NO: 36 - protein, 10 aa, Artificial Sequence
  Designed peptide
  MOD_RES (10)..(10): Carboxyl group of the C-terminus is amidated.
LSSTQAQQSY

SEQ ID NO: 37 - protein, 11 aa, Artificial Sequence
  Designed peptide
LSSTQAQQSYW

SEQ ID NO: 38 - protein, 11 aa, Artificial Sequence
  Designed peptide
WLSSTQAQQSY

SEQ ID NO: 39 - protein, 12 aa, Artificial Sequence
  Designed peptide
WLSSTQAQQSYW

SEQ ID NO: 40 - protein, 6 aa, Artificial Sequence
  Designed peptide
WLSSTQ

SEQ ID NO: 41 - protein, 6 aa, Artificial Sequence
  Designed peptide
AQQSYW

SEQ ID NO: 42 - protein, 14 aa, Artificial Sequence
  Designed peptide
ESFFLSSTQAQQSY

SEQ ID NO: 43 - protein, 14 aa, Artificial Sequence
  Designed peptide
LSSTQAQQSYLQGF

SEQ ID NO: 44 - protein, 14 aa, Artificial Sequence
  Designed peptide
FFLSSTQAQQSYLQ